(19) United States
(12) Patent Application Publication
(10) Pub. No.: US 2023/0311089 A1
(43) Pub. Date: Oct. 5, 2023

(54) PORTABLE DEVICE FOR MANUFACTURE OF PROTEIN-BASED DRUGS AND LABORATORY REAGENTS AND RELATED METHODS

(71) Applicant: THE GOVERNING COUNCIL OF THE UNIVERSITY OF TORONTO, Toronto (CA)

(72) Inventors: Hamed TINAFAR, Richmond Hill (CA); Keith PARDEE, Toronto (CA); David SINTON, Toronto (CA); Yuxiu GUO, Toronto (CA); Seray CICEK, York (CA); Soheil TALEBI, Toronto (CA)

(21) Appl. No.: 18/022,836

(22) PCT Filed: Aug. 26, 2021
(86) PCT No.: PCT/CA2021/051187

§ 371 Date: Feb. 23, 2023

Related U.S. Application Data
(60) Provisional application No. 63/070,336, filed on Aug. 26, 2020.

(51) Int. Cl. B01J 19/00; C07K 1/22

(52) U.S. Cl. CPC B01J 19/0046; C07K 1/22; B01J 2219/00315; B01J 2219/00459; B01J 2219/00596

(57) ABSTRACT

There are provided devices and methods for automated production of a purified target biomolecular product. The device can be portable, and can include a purification compartment, a wash buffer compartment, an elution buffer compartment, fluidic channels, a fluidic system and a processor powerable by a power source and operatively connected to the fluidic system for controlling the automated production of the purified target biomolecular product. The purification compartment can be configured to contain purification components and receive a raw biomolecular products mixture therein for binding a target biomolecular product and produce the purified target biomolecular product. The fluidic channels can be configured to successively introduce the raw biomolecular products mixture, a washing buffer and an elution buffer into the purification compartment.

### CROSS-REFERENCE TO RELATED APPLICATION

The present application claims priority from U.S. provisional patent application No. 63/070,336, filed on Aug. 26, 2020, and entitled “PORTABLE DEVICE FOR MANUFACTURE OF PROTEIN-BASED DRUGS AND LABORATORY REAGENTS AND RELATED METHODS”, the disclosure of which is hereby incorporated by reference in its entirety.

### TECHNICAL FIELD

The technical field generally relates to the production of biomolecular products, and more particularly to portable devices for manufacturing biomolecular products from cell-free systems.

### BACKGROUND

Manufacturing of protein-based products, such as vaccines and therapeutics, typically occurs in centralized, good manufacturing practice (GMP) approved facilities. The production of protein-based products is often controlled by regulatory agencies, which can impose stringent guidelines to ensure quality and suitability for human or animal use. Achieving a certain degree of purity through a series of standardized purification protocols for producing protein-based products is one of the main requirements. Most commonly used techniques in industry include traditional harvesting of proteins from a host organism and recombinant manufacturing of the proteins.

Traditional harvesting of proteins from a host organism, for which vaccine production can serve as an example, an immunogenic agent of a pathogen is identified through cumulative scientific research. Then, the pathogen is grown in large cultures and the immunogenic agent is purified by a process specific to the isolation of the agent.

Recombinant manufacturing techniques provide a methodology that can be applied to a variety of products, and can be used to purify proteins in laboratory settings. One way to carry out recombinant protein expression is to have a plasmid carrying the coding sequence for the protein of interest transformed/transfected into a host organism. The coding sequence can include a tag that allows specific binding of the protein of interest to an affinity matrix. Cells are then grown in a medium-rich media to favour production of the protein of interest, subsequently lysed and washed through a column containing a capture matrix. Beads are then washed to remove non-specific binding proteins and other impurities. Finally, the protein of interest can be eluted with a chemical agent, such as imidazole, or through enzymatic cleavage.

Recombinant manufacturing techniques using cells often involve certain challenges related to protein solubility, low yields of toxic proteins and a quality of the product that depends on the choice of host organism. Protein expression can result in inclusion bodies or preferred binding to insoluble cellular membrane. These proteins generally reside in an insoluble fraction that cannot be utilized for capture by the affinity matrix beads. Toxic proteins can also inhibit culture growth and result in poor yields. Finally, the choice of host organism can affect the type and availability of post-translational modifications resulting in unwanted products. As a result, these factors require testing and optimization in laboratory settings.

Cold chain distribution requirements can also contribute to a reduced access to protein-based therapeutics in remote or low resource settings when traditional protein manufacturing methods are utilized.

These aspects highlight drawbacks and challenges with respect to the production of protein-based products and the need for improved manufacturing and distribution techniques for the production of protein-based products.

### SUMMARY

In accordance with an aspect, there is provided a portable device for automated production of a purified target biomolecular product, the portable device comprising:
a water dispenser configured to contain water;
a cartridge-receiving section;
a cartridge configured to be received in the cartridge-receiving section, the cartridge comprising:
   a freeze-dried cell-free (FDCF) reaction compartment comprising FDCF reaction components activable upon rehydration via the water from the water dispenser to produce a raw biomolecular products mixture comprising a target biomolecular product and a measurable molecular reporter produced concomitantly with the target biomolecular product;
   a wash buffer compartment comprising wash components that are freeze-dried and produce a washing buffer upon rehydration via the water from the water dispenser;
   an elution buffer compartment comprising elution components that are freeze-dried and produce an elution buffer upon rehydration via the water from the water dispenser;
   a purification compartment comprising purification components that are freeze-dried and activable upon rehydration with the raw biomolecular products mixture, the purification components comprising an affinity matrix for binding the target biomolecular product, the purification compartment being configured to successively receive therein:
      the raw biomolecular products mixture to allow binding of the target biomolecular product to the affinity matrix and produce a biomolecular product mixture comprising a bound target biomolecular product;
      the washing buffer to wash the biomolecular product mixture and produce a waste mixture and a washed biomolecular product mixture comprising the bound target biomolecular product; and
      the elution buffer to subject the washed biomolecular product mixture to elution and produce the purified target biomolecular product; and
   a waste compartment configured to receive the waste mixture;
an optical tracking system for monitoring the production of the raw biomolecular product via the production of the measurable molecular reporter, the optical tracking system being configured to signal when the production of the measurable molecular reporter has reached a given threshold to indicate when to transfer the raw biomolecular products mixture to the purification compartment;
a pumping system for successively providing the raw biomolecular products mixture, the washing buffer and the elution buffer to the purification compartment, and for providing the water from the water dispenser to the FDCF reaction compartment, the wash buffer compartment, and the elution buffer compartment;
a tracing system to assign a unique identifier to a dose of the purified target biomolecular product;
a heating system operatively connected to the FDCF reaction compartment to provide heat thereto during the production of the raw biomolecular products mixture;
a cooling system operatively connected to the purification compartment to cool the same at least during the production of the bound target biomolecular product;
an actuator to initiate rehydration of the FDCF reaction components, wash components and elution components; and
a processor powerable by a power source and operatively connected to the optical tracking system, the pumping system, the tracing system, the heating system, the cooling system and the actuator for controlling the automated production of the purified target biomolecular product.

In accordance with another aspect, there is provided a portable device for automated production of a purified target biomolecular product, the device comprising:
a cartridge-receiving section;
a cartridge configured to be received in the cartridge-receiving section, the cartridge comprising:
   a cell-free reaction compartment configured to contain cell-free reaction components to produce a raw biomolecular products mixture comprising a target biomolecular product and a measurable molecular reporter produced concomitantly with the target biomolecular product;
   a wash buffer compartment configured to contain wash components and produce a washing buffer upon rehydration;
   an elution buffer compartment configured to contain elution components and produce an elution buffer upon rehydration;
   a purification compartment configured to contain purification components, the purification components comprising an affinity matrix for binding the target biomolecular product, the purification compartment being further configured to successively receive therein:
      the raw biomolecular products mixture to allow binding of the target biomolecular product to the affinity matrix and produce a biomolecular product mixture comprising a bound target biomolecular product;
      the washing buffer to wash the biomolecular product mixture and produce a waste mixture and a washed biomolecular product mixture comprising the bound target biomolecular product; and
      the elution buffer to subject the washed biomolecular product mixture to elution and produce the purified target biomolecular product; and
   a waste compartment configured to receive the waste mixture;
a tracking system for monitoring the production of the raw biomolecular product via the production of the measurable molecular reporter, the tracking system being configured to signal when the production of the measurable molecular reporter has reached a given threshold to indicate when to transfer the raw biomolecular products mixture to the purification compartment;
a pumping system for providing the raw biomolecular products mixture, the washing buffer and the elution buffer to the purification compartment;
a tracing system to assign a unique identifier to a dose of the purified target biomolecular product;
a heating system operatively connected to the cell-free reaction compartment to provide heat thereto during the production of the raw biomolecular products mixture;
a cooling system operatively connected to the purification compartment to cool the same at least during the production of the bound target biomolecular product;
an actuator to transfer or mix the cell-free reaction components, wash components and elution components; and
a processor operatively connected to the tracking system, the pumping system, the tracing system, the heating system, the cooling system or the actuator for controlling the automated production of the purified target biomolecular product.

In accordance with another aspect, there is provided a cartridge for use in a device for automated production of a purified target biomolecular product, the cartridge comprising:
a cell-free reaction compartment comprising cell-free reaction components to produce a raw biomolecular products mixture comprising a target biomolecular product and a measurable molecular reporter produced concomitantly with the target biomolecular product;
a wash buffer compartment comprising wash components for producing a washing buffer;
an elution buffer compartment comprising elution components for producing an elution buffer;
a purification compartment comprising purification components comprising an affinity matrix for binding the target biomolecular product, the purification compartment being configured to successively receive therein:
   the raw biomolecular products mixture to allow binding of the target biomolecular product to the affinity matrix and produce a biomolecular product mixture comprising a bound target biomolecular product;
   the washing buffer to wash the biomolecular product mixture and produce a waste mixture and a washed biomolecular product mixture comprising the bound target biomolecular product; and the elution buffer to subject the washed biomolecular product mixture to elution and produce the purified target biomolecular product.

In accordance with another aspect, there is provide a cartridge for use in a device for automated production of a purified target biomolecular product, the cartridge comprising:
a cell-free reaction compartment comprising cell-free reaction components to produce a raw biomolecular products mixture comprising a target biomolecular product and a measurable molecular reporter produced concomitantly with the target biomolecular product; and
a purification compartment comprising purification components comprising an affinity matrix for binding the target biomolecular product, the purification compartment being configured to successively receive therein:
   the raw biomolecular products mixture to allow binding of the target biomolecular product to the affinity matrix and produce a biomolecular product mixture comprising a bound target biomolecular product;
   a washing buffer to wash the biomolecular product mixture and produce a waste mixture and a washed biomolecular product mixture comprising the bound target biomolecular product; and
   an elution buffer to subject the washed biomolecular product mixture to elution and release the target biomolecular product from the affinity matrix to produce the purified target biomolecular product.

In accordance with another aspect, there is provided a cartridge for use in a device for automated production of a purified target biomolecular product, the cartridge comprising:
a cell-free reaction compartment comprising cell-free reaction components to produce a raw biomolecular products mixture comprising a target biomolecular product having a self-cleavage site and a measurable molecular reporter produced concomitantly with the target biomolecular product; and
a purification compartment comprising purification components comprising an affinity matrix for binding the target biomolecular product via an affinity tag, the purification compartment being configured to successively receive therein:
   the raw biomolecular products mixture to allow binding of the target biomolecular product to the affinity matrix via the affinity tag and produce a biomolecular product mixture comprising a bound target biomolecular product;
   a washing buffer to wash the biomolecular product mixture and produce a waste mixture and a washed biomolecular product mixture comprising the bound target biomolecular product; and
   an elution buffer to remove the target biomolecular product from the affinity matrix, the elution buffer comprising a self-cleaving initiator component to cleave the target biomolecular product at the cleavage site and produce the purified target biomolecular product.

In accordance with another aspect, there is provided a cartridge for use in a device for automated production of a purified target biomolecular product, the cartridge comprising:
a cell-free reaction compartment comprising cell-free reaction components to produce a raw biomolecular products mixture comprising a target biomolecular product having a cleavage site and a measurable molecular reporter produced concomitantly with the target biomolecular product; and
a purification compartment comprising purification components comprising a first affinity matrix for binding the target biomolecular product via an affinity tag and a second affinity matrix for binding the cleavage component once cleaved from the target biomolecular product, the purification compartment being configured to successively receive therein:
   the raw biomolecular products mixture to allow binding of the target biomolecular product to the first affinity matrix via the affinity tag and produce a biomolecular product mixture comprising a bound target biomolecular product;
   a washing buffer to wash the biomolecular product mixture and produce a waste mixture and a washed biomolecular product mixture comprising the bound target biomolecular product; and
   an elution buffer to remove the target biomolecular product from the first affinity matrix, the elution buffer comprising a cleavage component to cleave the target biomolecular product at the cleavage site to remove the affinity tag therefrom and allow binding of the cleavage component to the second affinity matrix to produce the purified target biomolecular product.

In accordance with another aspect, there is provided a portable device for automated production of a purified target biomolecular product, the portable device comprising:
a cartridge comprising a cell-free reaction compartment and a purification compartment, the cell-free reaction compartment comprising:
   cell-free reaction components to produce a raw biomolecular products mixture comprising a target biomolecular product and a measurable molecular reporter produced concomitantly with the target biomolecular product;
an optical tracking system for monitoring the production of the raw biomolecular product via the production of the measurable molecular reporter, the optical tracking system being configured to estimate the amount of biomolecular product being made in real time or to signal when the production of the measurable molecular reporter has reached a given threshold to indicate when to transfer the raw biomolecular products mixture to the purification compartment.

In accordance with another aspect, there is provided a portable device for automated production of a purified target biomolecular product, the portable device comprising:
a wet compartment section comprising:
   a cartridge-receiving section for receiving a cartridge that comprises:
   a cell-free reaction compartment comprising cell-free reaction components to produce a raw biomolecular products mixture comprising a target biomolecular product;
   a wash buffer compartment;
   an elution buffer compartment; and
   a purification compartment comprising purification components for binding the target biomolecular product and produce the purified target biomolecular product;
a dry compartment section separated from the wet compartment section, the dry component section comprising:
   a pumping system for providing the raw biomolecular products mixture, a washing buffer from the wash buffer compartment, and an elution buffer from the elution buffer compartment to the purification compartment;
   a heating system operatively connected to the cell-free reaction compartment to provide heat during the production of the raw biomolecular products mixture; and
   a processor operatively connected to the pumping system and the heating system for controlling the automated production of the purified target biomolecular product;
   electronic circuitry to connect the pumping system and the heating system to the processor; and
a barrier to physically separate the wet compartment section from the dry compartment section.

In accordance with another aspect, there is provided a portable device for automated production of a purified target biomolecular product, the portable device comprising:
a bottom wall, side walls extending from the bottom wall to define a reaction chamber, and a top wall;
an aqueous solution dispenser;
a cartridge-receiving section provided within the reaction chamber;
a cartridge configured to be received in the cartridge-receiving section, the cartridge comprising:
   cartridge compartments provided in a spaced-apart relationship from each other, the cartridge compartments comprising:
      a reaction compartment configured to produce a raw biomolecular products mixture comprising a target biomolecular product;
      a wash buffer compartment configured to contain wash components and produce a washing buffer upon rehydration;
      an elution buffer compartment configured to contain elution components and produce an elution buffer upon rehydration; and
      a purification compartment being configured to contain purification components to produce a purified target biomolecular product; and
   wherein the reaction compartment, the wash buffer compartment and the elution buffer compartment are configured to be in fluid communication with the aqueous solution dispenser once the top wall is closed for the rehydration to occur; and
   wherein the reaction compartment, the wash buffer compartment and the elution buffer compartment are configured to be in fluid communication with the purification compartment following rehydration and in accordance with pre-determined reaction time points;
an optical tracking system for monitoring the production of the target biomolecular product;
a pumping system for providing the raw biomolecular products mixture, the washing buffer, and the elution buffer to the purification compartment and for providing the aqueous solution from the aqueous solution dispenser to the reaction compartment, the wash buffer compartment, and the elution buffer compartment;
a heating system operatively connected to the reaction compartment to provide heat thereto;
an actuator to initiate rehydration of the reaction components, wash components and elution components once the top wall is closed; and
a processor operatively connected to the optical tracking system, the pumping system, the tracing system, the heating system and the actuator for controlling the automated production of the purified target biomolecular product.

In accordance with another aspect, there is provided a portable device for automated production of a purified target biomolecular product, the portable device comprising:
a bottom wall, side walls extending from the bottom wall to define a reaction chamber, and a top wall;
an aqueous solution dispenser adjacent to the reaction chamber;
a cartridge-receiving section provided within the reaction chamber;
a cartridge configured to be received in the cartridge-receiving section, the cartridge comprising:
   cartridge compartments comprising:
      a reaction compartment configured to produce a raw biomolecular products mixture comprising a target biomolecular product;
      a wash buffer compartment configured to contain wash components and produce a washing buffer upon rehydration;
      an elution buffer compartment configured to contain elution components and produce an elution buffer upon rehydration; and
      a purification compartment being configured to contain purification components to produce a purified target biomolecular product; and
   wherein the reaction compartment, the wash buffer compartment and the elution buffer compartment are configured to be in fluid communication with the aqueous solution dispenser once the top wall is closed for the rehydration to occur; and
   wherein the reaction compartment, the wash buffer compartment and the elution buffer compartment are configured to be in fluid communication with the purification compartment following rehydration and in accordance with pre-determined reaction time points.

In accordance with another aspect, there is provided a method for automated production of a purified target biomolecular product, the method comprising:
inserting a cartridge that comprises a cell-free reaction compartment, a wash buffer compartment, an elution buffer compartment, a purification compartment and a waste compartment into a cartridge-receiving section of a portable device;
rehydrating the cell-free reaction compartment comprising reaction components to produce a raw biomolecular products mixture comprising a target biomolecular product and a measurable molecular reporter produced concomitantly with the target biomolecular product;
rehydrating freeze-dried wash components within the wash buffer compartment to produce a washing buffer;
rehydrating freeze-dried elution components within the elution buffer compartment to produce an elution buffer;
subjecting the raw biomolecular products mixture to purification to produce the purified biomolecular product, comprising:
   transferring the raw biomolecular products mixture to the purification compartment, the purification compartment comprising purification components activable upon rehydration and configured to bind the target biomolecular product and produce a biomolecular product mixture comprising a bound target biomolecular product;
   transferring the wash buffer to the purification compartment to wash the biomolecular product mixture and produce a waste mixture and a washed biomolecular product mixture comprising the bound target biomolecular product;
   removing the waste product from the purification compartment and transferring the waste product to the waste compartment; and
   transferring the elution buffer to the purification compartment to elute the purified target biomolecular product;
wherein the automated production of the purified target biomolecular product is controlled by a processor.

In accordance with another aspect, there is provided a portable device for automated production of a purified target biomolecular product, the portable device comprising:
a cartridge-receiving section;
a cartridge configured to be received in the cartridge-receiving section, the cartridge comprising:
   a cell-free reaction compartment configured to contain cell-free reaction components therein to produce a raw biomolecular products mixture comprising a target biomolecular product; and
   a purification compartment configured to contain purification components and receive the raw biomolecular products mixture therein for binding the target biomolecular product and produce the purified target biomolecular product;
a pumping system for providing the raw biomolecular products mixture to the purification compartment; and
a processor powerable by a power source and operatively connected to the pumping system for controlling the automated production of the purified target biomolecular product.

In accordance with another aspect, there is provided a method for automated production of a purified target biomolecular product, the method comprising:
positioning a cartridge onto a cartridge-receiving section of a portable device, the cartridge comprising:
   a cell-free reaction compartment configured to contain cell-free reaction components; and
   a purification compartment configured to contain purification components;
producing a raw biomolecular products mixture comprising a target biomolecular product from the cell-free reaction components;
subjecting the raw biomolecular products mixture to purification to produce the purified biomolecular product;
wherein the automated production of the purified target biomolecular product is controlled by a processor.

In accordance with another aspect, there is provided a portable, modular platform for cell-free production, purification and formulation of a protein or RNA or a combination thereof, wherein the platform comprises:
a device that receives one or more loading modules, each loading module containing one or more reaction reagents relating to transcription or translation or a combination of both as well as one or more reagents required for purification;
wherein the loading module contains at least one reactor module and one purification module;
wherein reactor module facilitates production of a target protein or RNA or a combination thereof via a process selected from the group consisting of:
   continuous exchange cell-free (CECF) and
   batch cell-free synthesis;
wherein said reactor module may contain one or more chambers that store one or more of the reaction reagents relating to transcription or translation or a combination of both before or while the loading module is loaded onto the device; and
wherein said purification module includes one or more chromatography modules for separating one or more target protein, RNA or combination thereof from one or more components of the reaction solution;
wherein said purification module may contain one or more chambers or columns to store one or more of the purification reagents before or while the loading module is loaded onto the device.

In accordance with another aspect, there is provided a portable device for automated production of a purified target biomolecular product, the portable device comprising:
a purification compartment configured to contain purification components and to receive a raw biomolecular products mixture comprising a target biomolecular product, the purification components comprising an affinity matrix for binding the target biomolecular product;
a wash buffer compartment configured to contain a washing buffer comprising wash components;
an elution buffer compartment configured to contain an elution buffer comprising elution components;
fluidic channels configured to successively introduce into the purification compartment:
   the raw biomolecular products mixture to allow binding of the target biomolecular product to the affinity matrix and produce a biomolecular product mixture comprising a bound target biomolecular product;
   the washing buffer to wash the biomolecular product mixture and produce a waste mixture and a washed biomolecular product mixture comprising the bound target biomolecular product; and
   the elution buffer to subject the washed biomolecular product mixture to elution and produce the purified target biomolecular product;
a fluidic system comprising a pump to supply the raw biomolecular products mixture, the washing buffer and the elution buffer to the purification compartment; and
a processor operatively connected to the fluidic system via electronic circuitry to control operation of the fluidic system.

In some implementations, the fluidic system comprises valves that enable selective fluid communication of the wash buffer compartment and the elution buffer compartment with the purification compartment via the processor.

In some implementations, the selective fluid communication is in accordance with pre-determined time points of a pre-determined production protocol.

In some implementations, the fluidic channels are etched, carved, embossed or molded in a manifold plate.

In some implementations, the wash buffer compartment and the elution buffer compartment are each in fluid communication with a corresponding one of the fluidic channels via a corresponding fluidic port provided in the manifold plate.

In some implementations, the manifold plate comprises a compartments-receiving section, and at least one of the wash buffer compartment and the elution buffer compartment is coupled to the manifold plate via the compartments-receiving section.

In some implementations, the manifold plate comprises two etched sheets superposed to one another to obtain the fluidic channels.

In some implementations, the two etched sheets are etched acrylic sheets bonded together with a pressure adhesive, pressure controlled lamination, or temperature controlled lamination.

In some implementations, at least one of the purification compartment, the wash buffer compartment and the elution buffer compartment is removably coupled to the manifold plate via a corresponding fluidic port.

In some implementations, the fluidic channels are made of tubing.

In some implementations, the device further comprises a reaction compartment comprising reaction components configured to produce the raw biomolecular products mixture comprising the target biomolecular product.

In some implementations, the device further comprises a heating system operatively connected to the reaction compartment and the processor to provide heat to the reaction compartment during the production of the raw biomolecular products mixture.

In some implementations, the device further comprises a cooling system operatively connected to the processor and to at least one of the purification compartment and the reaction compartment.

In some implementations, the reaction compartment further contains a measurable molecular reporter produced concomitantly with the target biomolecular product.

In some implementations, the reaction components are cell-free reaction components.

In some implementations, the cell-free reaction components are freeze-dried cell-free (FDCF) reaction components activable upon rehydration to produce the raw biomolecular products mixture.

In some implementations, the cell-free reaction components enable production of RNA, purification of RNA, amplification of DNA, and/or purification of DNA.

In some implementations, the reaction components comprise DNA and/or RNA coding for the target biomolecular product that is provided as dried pellets.

In some implementations, the device further comprises an optical tracker in optical communication with the reaction compartment for monitoring the production of the raw biomolecular product via the production of the measurable molecular reporter.

In some implementations, the optical tracker is at least one of a colorimetric tracker, a fluorescent tracker, and a UV tracker.

In some implementations, the optical tracker is configured to signal the processor when the production of the measurable molecular reporter has reached a given threshold to indicate when to transfer the raw biomolecular products mixture to the purification compartment.

In some implementations, the measurable molecular reporter comprises a LacZ reporter gene, and the optical tracker is configured to monitor the production of the raw biomolecular product via the production of the LacZ reporter gene by measuring absorbance at a wavelength of approximately 570 nm.

In some implementations, the molecular reporter is a fluorescent reporter, and the fluorescent reporter is at least one of green fluorescent protein (GFP), enhanced green fluorescent protein (EGFP), mCherry and red fluorescent protein (RFP).

In some implementations, the device further comprises at least one of an enzymatic tracker, an affinity-based tracker, and an electrochemical tracker.

In some implementations, the device further comprises a binding buffer compartment in fluid communication with the purification compartment, the binding buffer compartment being configured to contain a binding buffer comprising buffer components.

In some implementations, the device further comprises a waste compartment in fluid communication with the purification compartment, the waste compartment being configured to receive waste components from the purification compartment.

In some implementations, the device further comprises a product dispensing compartment in fluid communication with the purification compartment, the product dispensing compartment being configured to receive the purified target biomolecule product from the purification compartment.

In some implementations, at least one of the wash components and the elution components are freeze-dried and activable upon rehydration.

In some implementations, the purification components are freeze-dried and activable upon rehydration with the raw biomolecular products mixture.

In some implementations, the affinity matrix comprises at least one of chitin-binding beads, nickel-nitrilotriacetic acid (Ni-NTA) beads, protein-A beads, amylose resin, agarose, and cellulose matrices, or combination thereof.

In some implementations, the elution components comprise an enzymatic cleavage component configured to release the target biomolecular product from the affinity matrix.

In some implementations, the enzymatic cleavage component is a TEV protease or a chitin-binding/GST-tag/TEV Protease (S219V) chimera.

In some implementations, the elution components comprise a self-cleaving initiator component configured to release the target biomolecular product from the affinity matrix.

In some implementations, the self-cleaving initiator component comprises at least one of dithiothreitol (DTT), 2-mercaptoethanol, cysteine, and hydroxylamine.

In some implementations, the elution components comprise a chemical agent configured to release the target biomolecular product from the affinity matrix.

In some implementations, the chemical agent comprises imidazole or Histidine.

In some implementations, the device further comprises a tracing system operatively connected to the processor, the tracing system configured to assign a unique identifier to a dose of the purified target biomolecular product.

In some implementations, the device further comprises an actuator operatively connected to the processor via the electronic circuitry to initiate fluid communication between the washing buffer compartment and the purification compartment, or between the elution buffer compartment and the purification compartment.

In some implementations, the purified target biomolecular product comprises a therapeutic drug, a vaccine, an antibody or antibody fragment, a protein-based reagent, or an antivenom.

In some implementations, the portable device is configured to collect a series of manufacturing metrics.

In some implementations, the series of manufacturing metrics is storable in an immutable block chain ledger.

In some implementations, the series of manufacturing metrics comprises at least one of a date, a reaction duration, a reaction temperature, an origin of the cartridge, a DNA sequence, a yield of the purified target biomolecular product, location of manufacturing, identity of user, and a quality metric.

In some implementations, the device further comprises an on-site DNA synthesizer.

In some implementations, at least one of the washing buffer compartment, the elution buffer compartment, and the purification compartment is an individual tube or vial.

In some implementations, the washing buffer compartment, the elution buffer compartment, and the purification compartment are sealed and segmented compartments and are provided as part of a cartridge.

In some implementations, the sealed and segmented compartments are configured to be punctured.

In some implementations, the device further comprises a reaction compartment comprising reaction components configured to produce the raw biomolecular products mixture comprising the target biomolecular product.

In some implementations, the reaction compartment is configured to be punctured upon closing a lid of the portable device, loading the cartridge or activation of an actuator.

In some implementations, the sealed and segmented compartments are punctured simultaneously or according to a predetermined order.

In some implementations, the device further comprises a pressure sensor operatively connected to the processor to monitor a pressure within at least one of the washing buffer compartment, the washing buffer compartment, and the purification compartment, and/or within the fluidic channels extending therebetween.

In some implementations, the device further comprises a flow sensor operatively connected to the processor to monitor a flow rate between the washing buffer compartment and the purification compartment, or between the elution buffer compartment and the purification compartment.

In some implementations, the processor is separated from wet components of the portable device to prevent passage of liquid to the processor, the wet components comprising at least one of the compartments.

In some implementations, the device further comprises a second optical tracker in optical communication with the fluidic channels for monitoring fluid circulation within the fluidic channels.

In accordance with another aspect, there is provided a method for automated production of a purified target biomolecular product from a raw biomolecular products mixture comprising a target biomolecular product, the method comprising:
activating a portable device to initiate the automated production of the purified target biomolecular product, the portable device comprising:
   a purification compartment comprising purification components configured to bind the target biomolecular product;
   a fluidic system configured to supply the raw biomolecular products mixture to the purification compartment; and
   a processor to control the automated production of the purified target biomolecular product; and
subjecting the raw biomolecular products mixture to purification to produce the purified biomolecular product.

In some implementations, subjecting the raw biomolecular products mixture to purification comprises:
supplying the raw biomolecular products mixture to the purification compartment to produce a biomolecular product mixture comprising a bound target biomolecular product;
transferring a wash buffer to the purification compartment to wash the biomolecular product mixture and produce a waste mixture and a washed biomolecular product mixture comprising the bound target biomolecular product;
removing the waste product from the purification compartment; and
transferring an elution buffer to the purification compartment to elute the purified target biomolecular product.

In some implementations, the portable device further comprises a washing buffer compartment configured to contain the wash buffer.

In some implementations, the portable device further comprises an elution buffer compartment configured to contain the elution buffer.

In some implementations, the method further comprises synthesizing the raw biomolecular products mixture comprising a target biomolecular product and a measurable molecular reporter produced concomitantly with the target biomolecular product in a reaction compartment prior to supplying the raw biomolecular products mixture to the purification compartment.

In some implementations, the method further comprises activating the fluidic system to pump air into the reaction compartment during synthesis of the raw biomolecular products mixture.

In some implementations, the method further comprises monitoring a temperature of the reaction components in the reaction compartment via a temperature sensor.

In some implementations, the method further comprises adjusting the temperature in the reaction compartment with a heating system and/or a cooling system.

In some implementations, adjusting the temperature in the reaction compartment is performed to maintain reaction conditions according to a pre-determined protocol.

In some implementations, the method further comprises monitoring the production of the raw biomolecular product via the production of the measurable molecular reporter.

In some implementations, monitoring of the production of the raw biomolecular product via the production of the measurable molecular reporter is performed to produce a single dose of the purified target biomolecular product or a plurality of doses thereof.

In some implementations, monitoring the production of the raw biomolecular product via the production of the measurable molecular reporter allows for batch number traceability.

In some implementations, the portable device further comprises a product dispenser compartment.

In some implementations, the method further comprises monitoring a temperature of the purified target biomolecular product in the product dispenser compartment via a temperature sensor.

In some implementations, the method further comprises adjusting the temperature in the product dispenser compartment with a heating system and/or a cooling system.

In some implementations, adjusting the temperature in the product dispenser compartment is performed to maintain stabilizing conditions according to a pre-determined validation protocol.

In some implementations, the method further comprises transferring a binding buffer from a binding buffer compartment to the purification compartment to equilibrate the purification components prior to supplying the raw biomolecular products mixture to the purification compartment.

In some implementations, the method further comprises monitoring a pressure within at least of the washing buffer compartment, the elution buffer compartment, the purification compartment, and/or within the fluidic channels extending therebetween.

In some implementations, the method further comprises monitoring a flow rate of the components between the washing buffer compartment and the purification compartment, or between the elution buffer compartment and the purification compartment.

In accordance with another aspect, there is provided a cartridge for use in a device for automated production of a purified target biomolecular product, the cartridge comprising:
a reaction compartment comprising a raw biomolecular products mixture comprising a target biomolecular product and a measurable molecular reporter; and
a purification compartment comprising purification components, the purification compartment being configured to successively receive therein:
   the raw biomolecular products mixture to allow binding of the target biomolecular product to produce a bound target biomolecular product;
   a washing buffer to wash the bound target biomolecular product and produce a waste mixture and a washed biomolecular product mixture; and
   an elution buffer to subject the washed biomolecular product mixture to elution and release the target biomolecular product from the purification components to produce the purified target biomolecular product.

In some implementations, the purification components comprise an affinity matrix for binding the target biomolecular product.

In some implementations, the cartridge further comprises a washing buffer compartment configured to contain the washing buffer.

In some implementations, the cartridge further comprises an elution buffer compartment configured to contain the elution buffer.

In some implementations, the reaction compartment comprises reaction components to produce the raw biomolecular products mixture comprising the target biomolecular product and the measurable molecular reporter produced concomitantly with the target biomolecular product.

In accordance with another aspect, there is provided a portable device for automated production of a purified target biomolecular product, the portable device comprising:
compartments coupled to a compartments-receiving section, the compartments comprising:
   a wash buffer compartment configured to contain wash components;
   an elution buffer compartment configured to contain elution components; and
   a purification compartment configured to contain purification components;
a fluidic system for successively providing a raw biomolecular products mixture, the washing buffer, and the elution buffer to the purification compartment; and
a processor operatively connected to the fluidic system via electronic circuitry to control operation of the fluidic system.

In accordance with another aspect, there is provided a portable device for automated production of a purified target biomolecular product, the portable device comprising:
a cartridge comprising:
   a purification compartment configured to contain purification components and to receive a raw biomolecular products mixture comprising a target biomolecular product for binding the target biomolecular product;
   a wash buffer compartment configured to contain a washing buffer comprising wash components; and
   an elution buffer compartment configured to contain an elution buffer comprising elution components;
a fluidic system for successively providing a raw biomolecular products mixture, the washing buffer, and the elution buffer to the purification compartment; and
a processor powerable by a power source and operatively connected to the fluidic system for controlling the automated production of the purified target biomolecular product.

In some implementations, the device further comprises one or more features as defined herein and/or as described herein and/or illustrated herein.

In some implementations, the method further comprises one or more features as defined herein and/or as described herein and/or illustrated herein.

### BRIEF DESCRIPTION OF THE DRAWINGS

The attached figures illustrate various features, aspects and implementations of the technology described herein.

FIG. 1 shows a perspective view of a portable device for automated production of a purified target biomolecular product, the portable device being shown in an open configuration, and a cartridge for use therein, according to an implementation.

FIG. 2 shows a front view of the portable device shown in FIG. 1 in a closed configuration.

FIG. 3 shows a perspective view of a cartridge for use in the portable device shown in FIG. 1.

FIG. 4 shows a flow chart depicting steps involved in the automated production of a purified target biomolecular product using a portable device described herein, according to an implementation.

FIG. 5 shows a flow chart depicting steps involved in the automated production of a purified target biomolecular product using a portable device described herein, according to an implementation.

FIG. 6A shows a perspective view of a portable device for automated production of a purified target biomolecular product, the portable device being shown in an open configuration and a cartridge for use therein, according to an implementation.

FIG. 6B shows a perspective view of the portable device shown in FIG. 6A in a closed configuration.

FIG. 6C shows a perspective view of the portable device shown in FIG. 6A in an open configuration and a syringe for delivery to a user.

FIG. 7A shows a perspective view of a portable device for automated production of a purified target biomolecular product, the portable device being shown in an open configuration, according to an implementation.

FIG. 7B shows a perspective view of a cartridge in a first stage for use in the portable device of FIG. 7.

FIG. 7C shows a perspective view of the cartridge shown in FIG. 7A in a second stage of production.

FIG. 7D shows a perspective view of the cartridge shown in FIG. 7A in a third stage of production.

FIG. 7E shows a perspective view of the cartridge shown in FIG. 7A in a fourth stage of production.

FIG. 8 shows an illustration of a cell-free reaction for the synthesis of a raw biomolecular products mixture.

FIG. 9 shows an example of electronic transfer of DNA sequences encoding instructions for manufacturing a target biomolecular product.

FIG. 10A shows a perspective view of a lid and body of a cartridge in a coupled configuration.

FIG. 10B shows a top view of the lid and body shown in FIG. 1 in an uncoupled configuration.

FIG. 100 shows a perspective view of the lid and body shown in FIG. 1 in an uncoupled configuration.

FIG. 11 shows an illustration of decentralized cell-free manufacturing of a target biomolecular product.

FIG. 12 shows an illustration of an antivenom plasmid structure.

FIG. 13 shows an illustration of TEV-based purification.

FIG. 14A shows a perspective view of a portable device in a closed configuration, according to another implementation.

FIG. 14B shows a top view of the portable device of FIG. 14A.

FIG. 14C shows a side view of the portable device of FIG. 14A.

FIG. 14D shows a front view of the portable device of FIG. 14A.

FIG. 15A shows a perspective view of a portable device according to another implementation.

FIG. 15B shows a perspective view of a bottom of a housing of the portable device shown in FIG. 15A to illustrate internal components of the portable device.

FIG. 16 shows a top view of a manifold plate for use in the portable device of FIGS. 14A to 15B.

FIG. 17 shows a perspective view of a fluidic port for use in the manifold plate shown in FIG. 16.

FIG. 18 shows a perspective view of an adaptor for use in the fluidic port shown in FIG. 17.

FIG. 19 shows a top view of a pump for use in the manifold plate shown in FIG. 16.

FIG. 20A shows a perspective view of a portable device for automated production of a purified target biomolecular product, according to another implementation.

FIG. 20B shows a front view of the portable device of FIG. 20A, with a purification compartment provided above a top surface a of manifold plate.

FIG. 21 shows a side view of an optical tracker for use in the portable device of FIG. 20.

FIG. 22 shows a chart illustrating measurements taken from the optical tracker of FIG. 21.

FIG. 23A shows a perspective view of an optical tracker, according to another implementation.

FIG. 23B shows a perspective view of the optical tracker of FIG. 23A prior to use.

FIG. 23C shows a perspective view of the optical tracker of FIG. 23A in use.

FIG. 23D shows a perspective view of the optical tracker of FIG. 23A in a closed configuration.

FIG. 24 shows a perspective view of an optical tracker, according to another implementation.

FIG. 25 shows a flow chart depicting steps involved in the automated production of a purified target biomolecular product using a portable device described herein, according to another implementation.

### DETAILED DESCRIPTION

Techniques described herein relate to portable devices for automated production of a purified target biomolecular product, and optionally to a cartridge for use in some implementations of such portable devices. Examples of target biomolecular products that can be produced include for instance protein-based therapeutic products such as drugs, vaccines or antivenoms that are suitable for administration to a human or an animal, or other types of protein-based reagents such as laboratory protein-based reagents.

The portable device can include various hardware components. In implementations where the portable device is to be used with a cartridge, the portable device can include a cartridge-receiving section, which can be located within a reaction chamber, to receive the cartridge. In other implementations, the portable device can include fluidic channels that can be provided in a manifold plate to establish fluid communication between various compartments of the portable device that include components involved in the production of the biomolecular product.

In some implementations, the portable device can be provided as a portable casing or benchtop casing defining a reaction chamber that is configured to receive the hardware components and, optionally, the cartridge. The casing can include a bottom wall and side walls extending from the bottom wall, and a top wall is hingedly connected to one of the side walls and that can be opened and closed by the user when needed, in particular to insert the cartridge in the reaction chamber and to remove the cartridge therefrom. Other configurations of the casing are also possible. In some implementations, the production of the target biomolecular product can be initiated following closure of the hinged wall, and optionally upon activation of an actuator, and the user is not required to be involved in the production of the target biomolecular product until the production is completed.

The cartridge includes various cartridge compartments, which can also be referred to as modules or loading modules, containing respective components suitable for production of the target biomolecular product. One of the compartments is a reaction compartment, which can be configured to include liquid, frozen or freeze-dried cell-free (FDCF) reaction components that can be used directly or that are activable upon rehydration. Another compartment is a purification compartment that is configured to include purification components, for instance liquid, frozen or freeze-dried purification components, which can also be activable upon rehydration. Other compartments can include, among others, a wash buffer compartment, an elution buffer compartment, and a waste compartment. The wash buffer compartment is configured to contain liquid, frozen or freeze-dried wash components that produce a wash buffer upon rehydration, and the elution buffer compartment is configured to include elution components that are in liquid form or that can produce an elution buffer upon rehydration. The various compartments of the cartridge can be isolated from one another to allow given reactions to occur therein and to store the wash buffer and the elution until they are ready for use. Fluid communication between selected compartments can also be established, via an automated process, for transferring a mixture contained in a given compartment to another given compartment when needed, or from and to vessels located outside of the cartridge.

In some implementations, an aqueous solution dispenser, such as a water dispenser, can be provided within the reaction chamber, or in proximity thereof, to supply an aqueous solution, such as water, to a given compartment when rehydration of components within the given compartment is needed, which can also be controlled via the automated process.

Hardware components of the portable device include a tracker, such as an optical tracker, a pumping system, which can also be referred to as a fluidic system, a heating system, a cooling system and a processor. The optical tracker is configured to monitor the production of the target biomolecular product to indicate when a desired production threshold is reached. The pumping system, which can also be referred to as a fluidic system, can include one or more pumps and associated valves to enable the addition of water from the water dispenser to given compartments and/or the transfer of fluids from a given compartment to another. The fluidic system may include a pressure sensor to monitor pressure within compartments and/or within fluidic connections therebetween, to give feedback to the device on adjusting the speed of the fluidic system. For instance, if the pressure is too high, the pumping rate can be lowered. Similarly, the fluidic system may include a flow sensor or flow meter to measure the flow rate within the fluidic system and provide feedback to the device to adjust the pump rate. The heating and the cooling systems are provided to allow reactions to occur at given temperatures. The heating and/or cooling system can include a temperature sensor to monitor the temperature of the reaction compartment, the purification compartment and/or the product dispenser compartment. For instance, the heating system can provide heat to the cell-free reaction compartment during the production of the target biomolecule product to maintain the fluid contained therein from about 10° C. to about 40° C. It may also be desired to cool some of the compartments, for instance to maintain stability of the protein components and/or in accordance with optimized reaction conditions.

The processor is configured to control the various steps of the production of the purified target biomolecular product by being operatively connected through electronic circuitry to at least one of the optical tracker, the fluidic system, the heating system and the cooling system.

In addition, a tracing system can be provided to enable the assignment of a unique identifier to a batch of the purified target biomolecular product that is produced, the unique identifier being determined in accordance with the characteristics of the cartridge that has been used for the production purified target biomolecular product. In turn, the unique identifier can provide traceability for doses of the purified target biomolecular product, and can allow to retain information regarding that batch, such as the duration and temperature of the reaction (either required or applied), the origin of cartridge, the DNA sequence used, and the yield.

When a user is ready to produce a target biomolecular product, a non-specific cartridge for the purification of a target biomolecular product may be used. In other applications a cartridge that is specific to that target biomolecular product, i.e., that contains cell-free reaction components, such as FDCF reaction components, designed to produce that specific target biomolecular product, is placed onto the cartridge-receiving section and the hinged wall of the portable device is closed. The production of the target biomolecular product can be initiated directly upon closure of the hinged wall, or the user can initiate the production of the target biomolecular product via an actuator that is operatively connected to the processor. The various automated steps that occur thereafter can be controlled via the processor up until the purified target biomolecular product is ready for use, without having to involve technical expertise from the user and without additional input from the user up until the dose of the purified target biomolecular product is ready for retrieval.

Various implementations of the portable device and associated methods will now be described in greater detail.

### Description of the Portable Device

The portable device described herein allows for the automated production of various biomolecular such as protein-based biomolecules, RNA, DNA, and combinations of biomolecules including formulations where a biomolecule is loaded into liposomes or combined with any other reagent used for drug delivery. The device is configured as a portable device to improve access to protein-based reagents and other biomolecules, and reduces or removes the requirement for technical expertise from the user. In some implementations, the portable device can be designed as having a volume of less than 10 litres, 5 litres, 2 litres or 1 litre, i.e., 10 000 cm³, 5 000 cm³, 2 000 cm³ or 1 000 cm³, and can be powered by a wall outlet or with a battery.

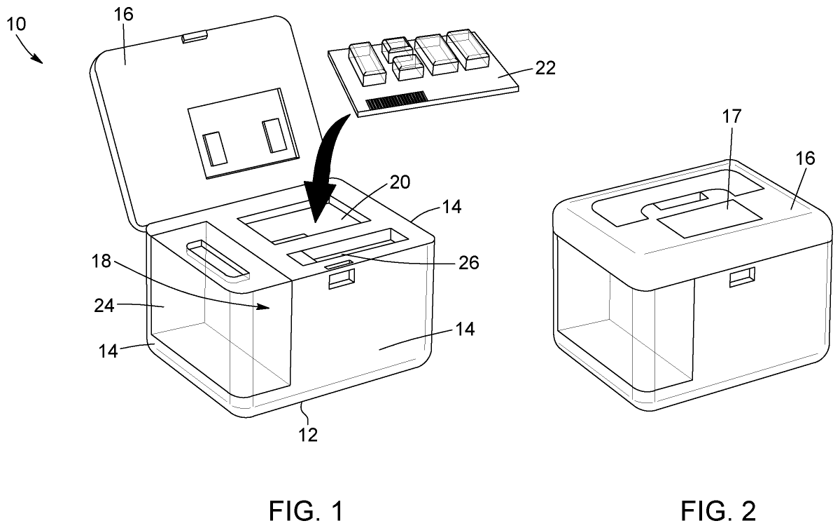

Referring now to FIG. 1, an example of a portable device 10 according to one implementation is shown in an open configuration. The housing of the portable device 10 includes a bottom wall 12, side walls 14 and a top wall 16 that hingedly connects to one of the side walls 14 such that the top wall 16 can alternate between being in an opened configuration such as shown in FIG. 1, to being in a closed configuration, such as shown in FIG. 2. The bottom wall 12 and the side walls 14 together define a reaction chamber 18. The reaction chamber 18 includes a cartridge-receiving section 20 that is configured to house a cartridge 22 that contains reaction components for the production of a purified target biomolecular product. In the implementation show, the portable device 10 also includes a section for receiving a water dispenser 24, the water dispenser 24 being provided outside and alongside one of the side walls 14 of the portable device 10. Alternatively, the water dispenser 24 can be provided within the reaction chamber 18, outside of the device or within the cartridge 22. In some implementations, the portable device 10 includes an indicator or control screen 17 that can be configured to display information relative to the progression of the automated production of the purified target biomolecular product or to take in custom user information and instructions.

When production of the purified target biomolecular product is completed, the user can retrieve a dose of the purified target biomolecular product from a product dispenser compartment 26. Alternatively, the dose of the purified target biomolecular product can be retrieved directly from the cartridge 22.

The reaction chamber 18 is configured to house various hardware components of the portable device 10, such as an optical tracker, a fluidic system, a heating system, a cooling system, a tracing system, a control unit or corresponding control units, and a processor (not shown). More detail regarding the hardware components provided within the reaction chamber 18 are described below.

### Cartridge

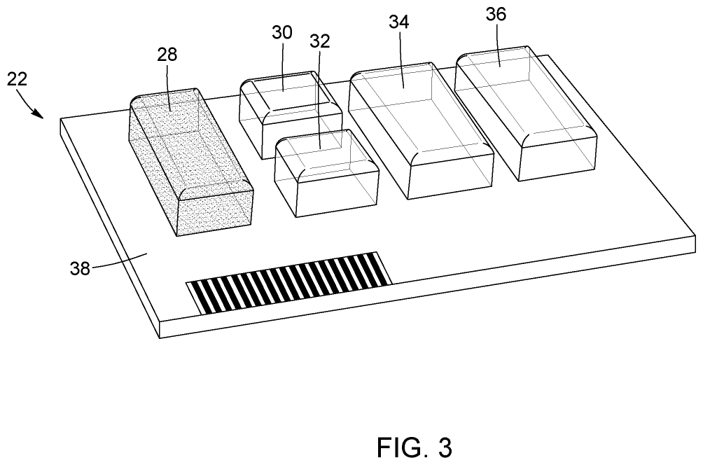

With reference now to FIG. 3, the cartridge 22 includes a cell-free reaction compartment 28, such as a FDCF reaction compartment, a wash buffer compartment 30, an elution buffer compartment 32, a waste compartment 34 and a purification compartment 36 that are provided onto a base 38. Alternatively, multiple compartments containing the various components for conducting the cell-free reaction can also be provided. In some implementations, the cartridge 22 can include a binding buffer compartment to equilibrate the beads before the cell-free reaction is put in contact with the beads. In the implementation shown, each of the compartments includes side walls that extend upwardly from the base 38 of the cartridge 22. In other implementations, one or more of the compartments can be provided according to another spatial arrangement than the one illustrated in FIG. 3. In some implementations, more than one cartridge 22 can be provided to supply the various compartments. In other words, compartments can be provided using separate cartridges. For instance, a second cartridge can be used to provide an additional purification compartment. The addition of a second purification compartment can depend on the reactions that are desired to occur in the automated process. Alternatively, the second purification compartment can also be provided on the cartridge 22 as well. Furthermore, in some implementations, a first cartridge including cell-free reaction compartment, such as a FDCF reaction compartment, can be provided, and a second cartridge including a purification compartment can be provided, with a wash buffer compartment and an elution buffer compartment being provided on either one of the first and second cartridges or on a third cartridge. It is to be understood that the number of compartments, their spatial organization, and the number of cartridges can vary, and that the techniques described herein are not limited to particular configurations of the cartridge.

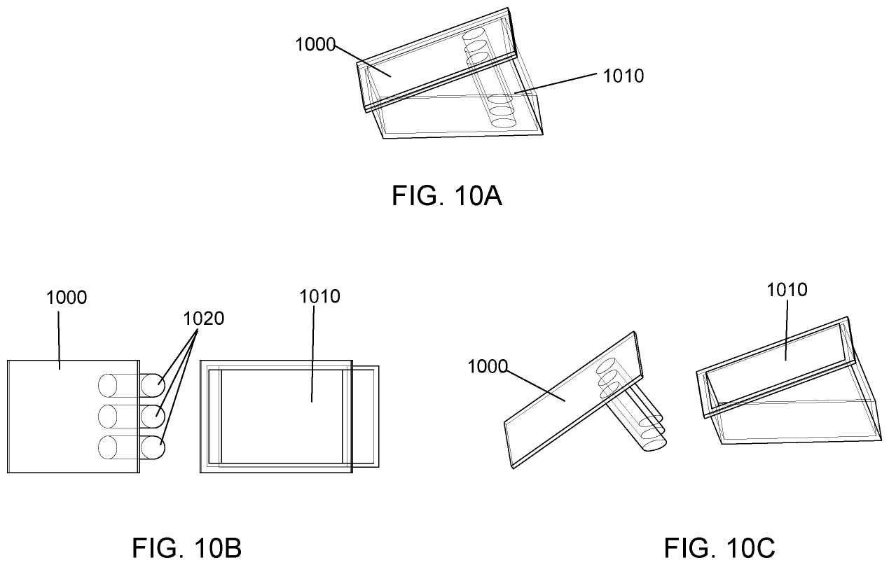

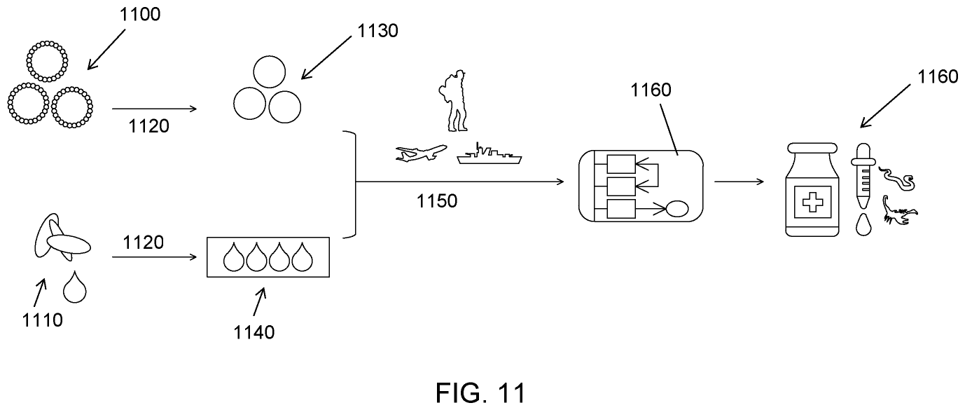

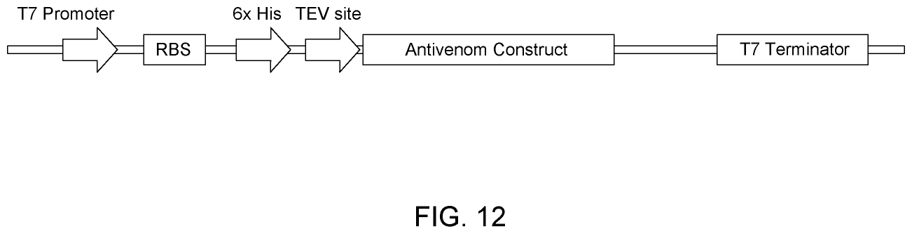

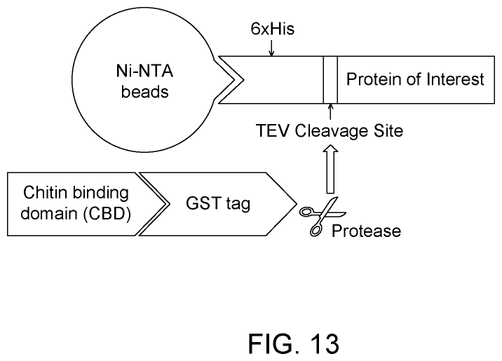

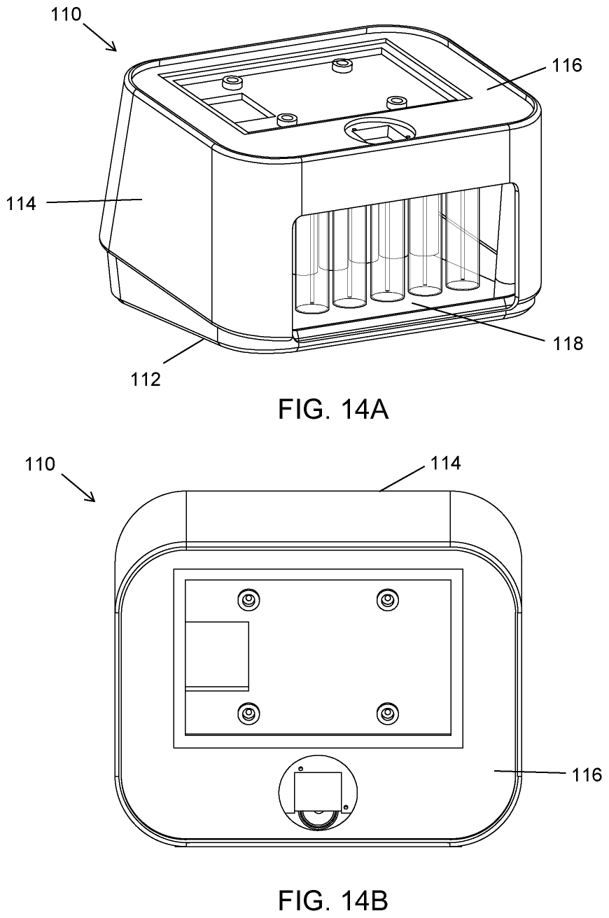

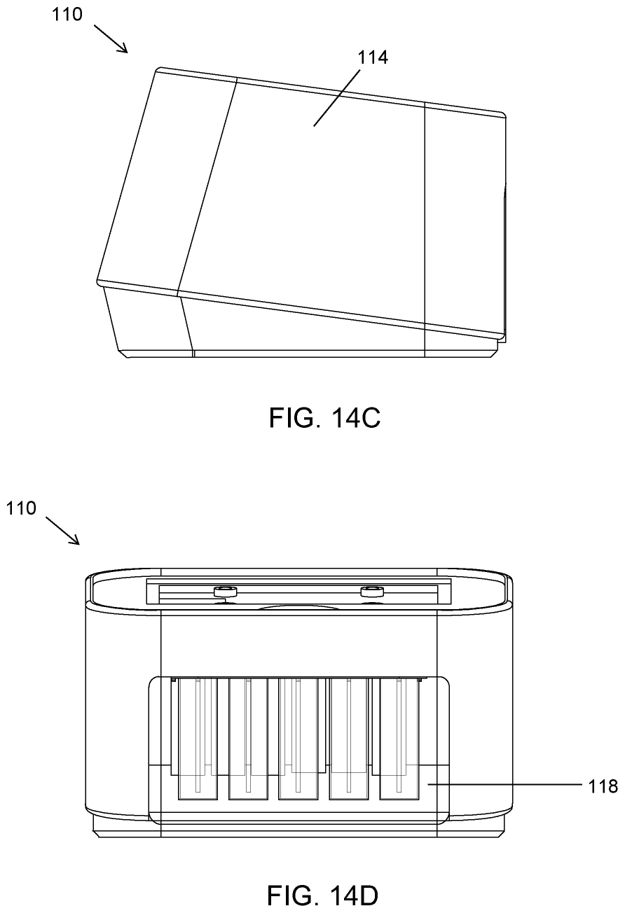

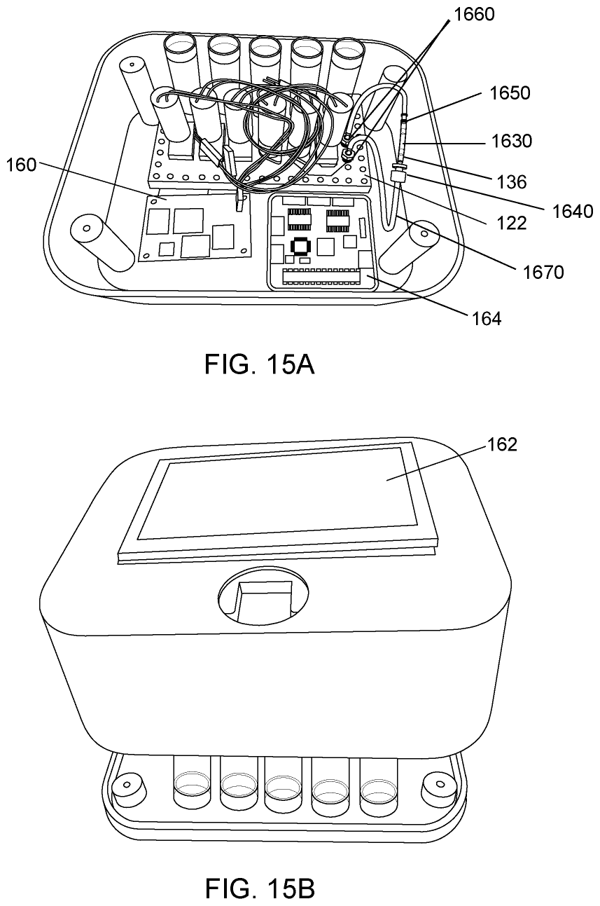

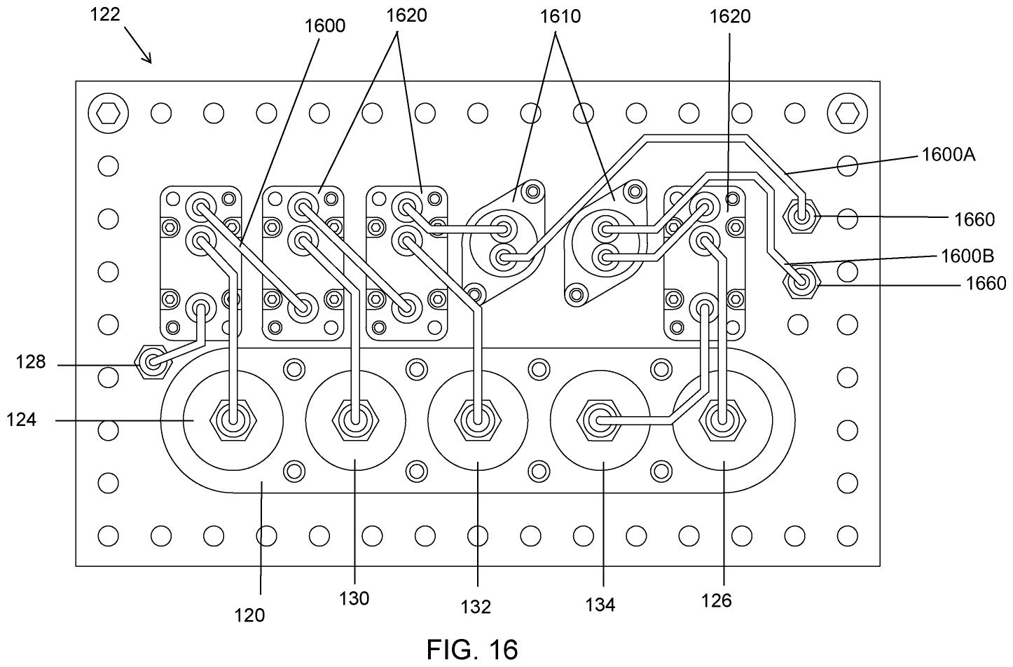

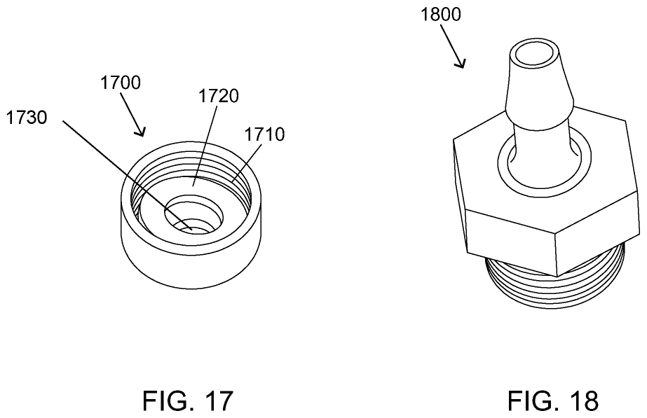

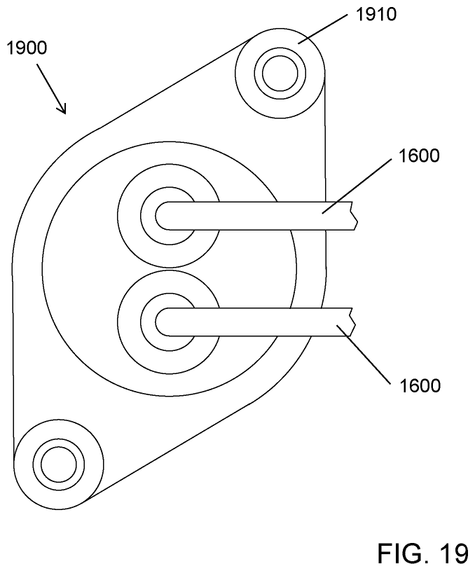

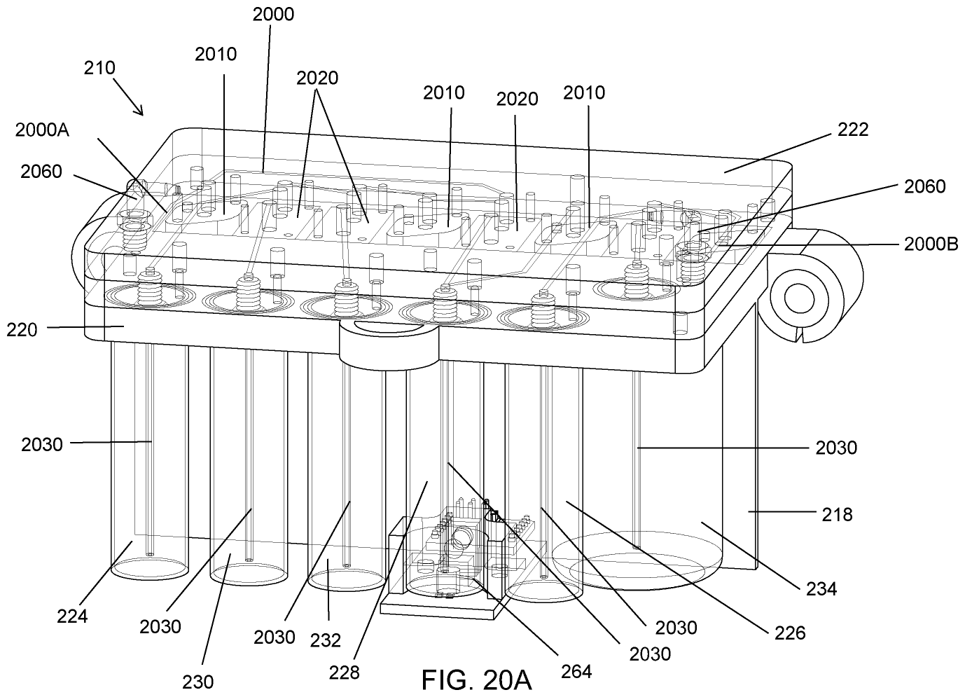

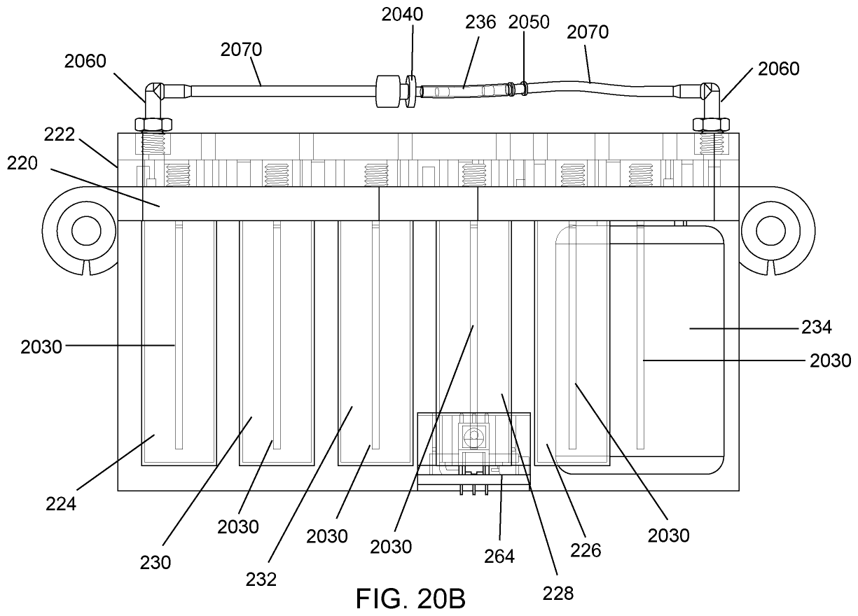

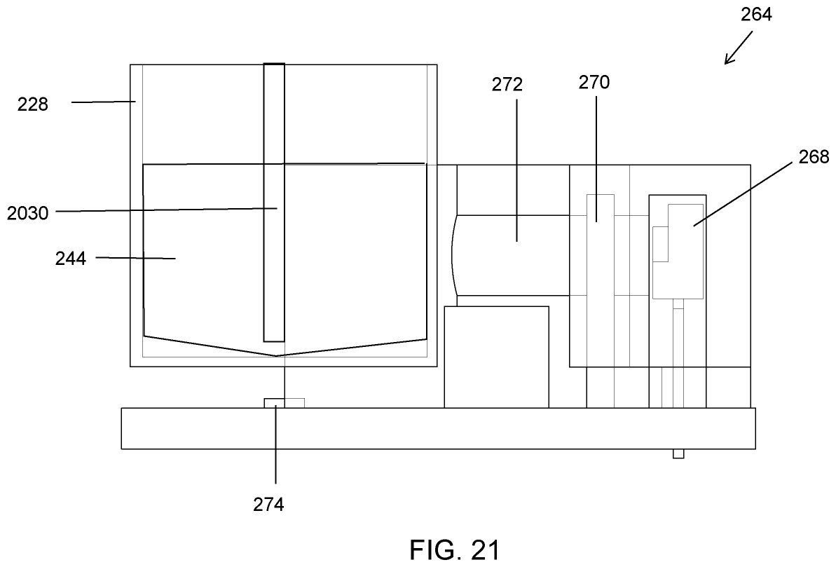

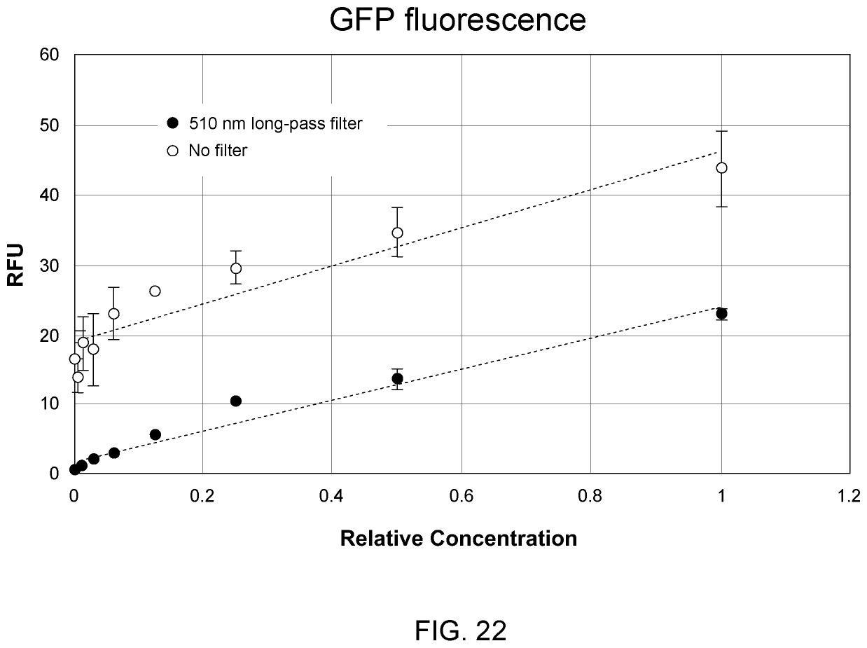

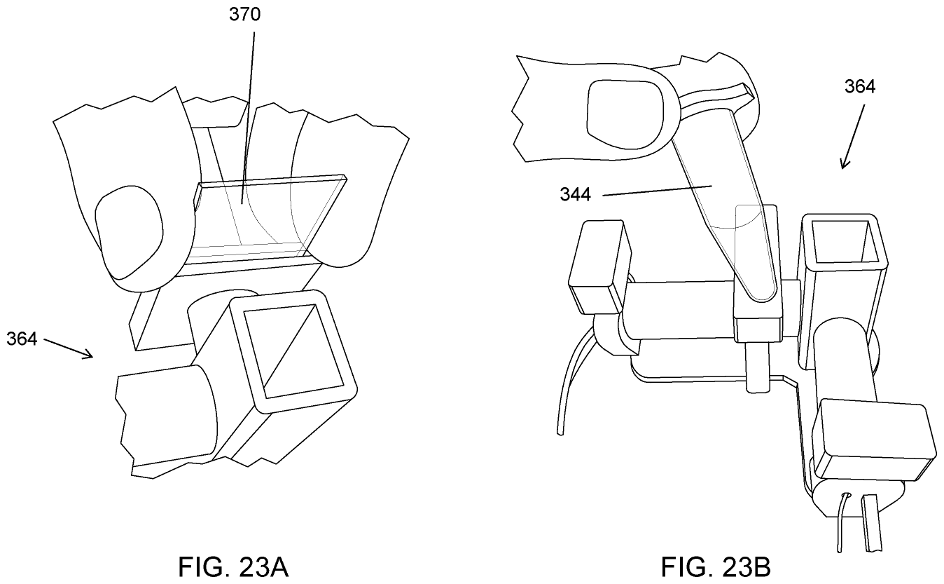

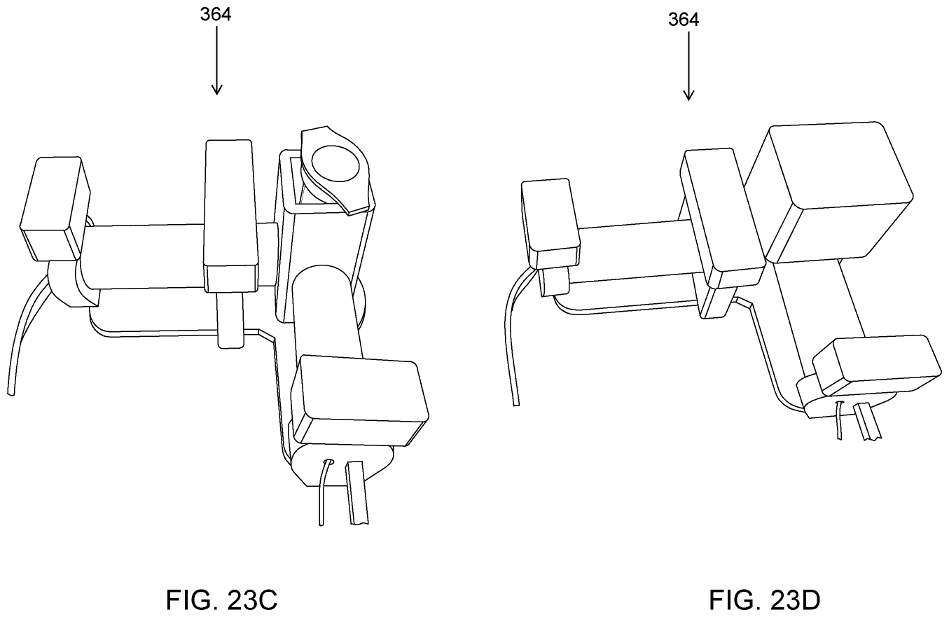

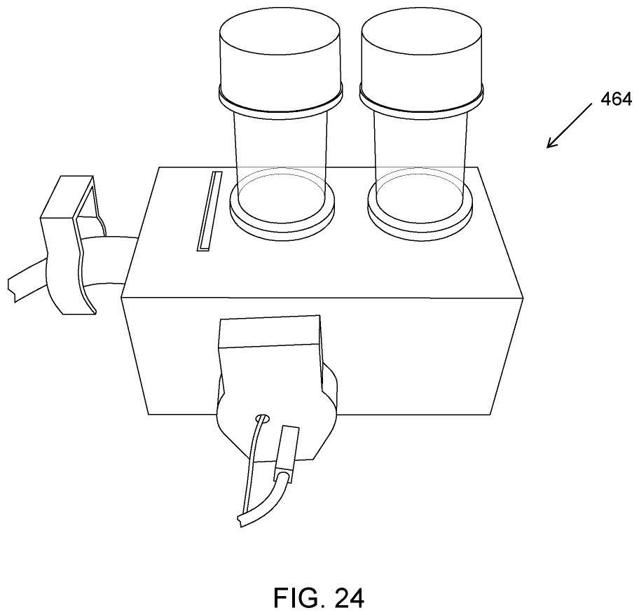

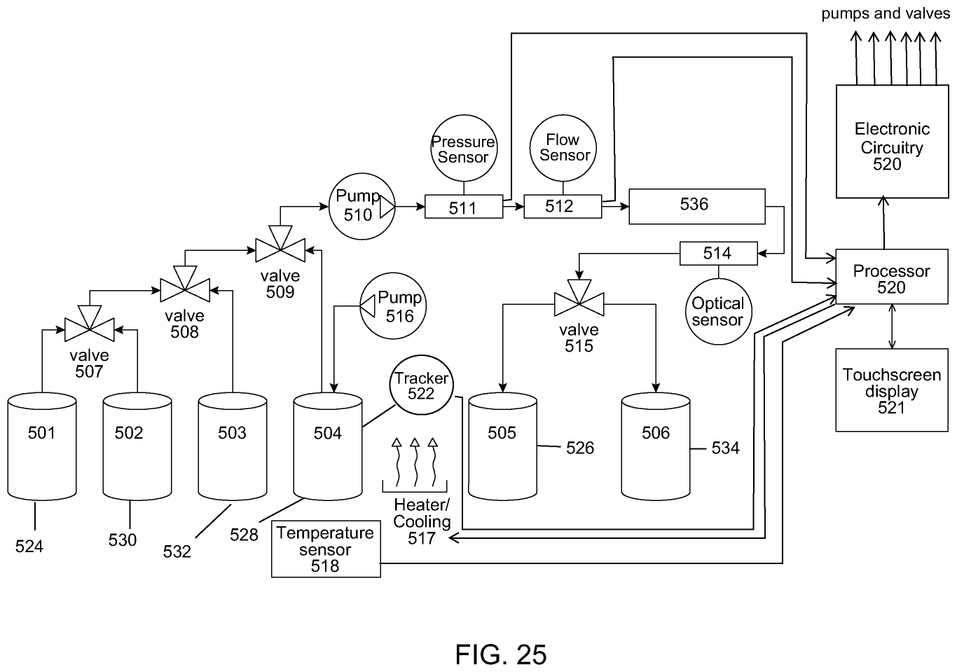

Furthermore, in some implementations, the base wall 38 can be omitted, and the various compartments can be provided as single units such as shown in FIGS. 10A to 100. In FIGS. 10A to 100, each compartment includes a lid 1000 and a body 1010. The lid 1000 can include a plurality of channels 1020, and can be configured to allow ports to enter and exit each compartment, which in turn can allow for transfer of components. The lid 1000 can also be configured to be punctured to enable fluid communication between different compartments of the cartridge 22 to occur, such as by being made of a material that can enable such puncture to occur. In some implementations, the lid 1000 can be integral with the body 1010 of the compartment, or the lid 1000 can be a separate component from the rest of the compartment, i.e., from the body 1010 of the compartment. In some implementations, the lid 1000 can be heated to minimize condensation on the top of the cartridge 22. The compartments can be provided in a spaced-apart relationship, for instance to provide the automated process to be sequential, and to avoid incompatibility issues between components of given compartments. The compartments can thus be sealed until addition or extraction of a given fluid is required according to the automated process, and can be configured to be punctured at a given time point during the automated process to allow fluid communication between selected compartments to be established.

The cartridge 22 can be configured to be pre-loaded with the various reaction components that are to be used in the production of the target biomolecular product. The various reaction components can be provided as dried reaction components to enhance their stability. In some implementations, the reaction components can be provided as freeze-dried components. Each cartridge 22 can be non-specific for the purification of a target biomolecular product or specifically designed for the production of a given target biomolecular product, in accordance with the input of the automated process controlling the steps of the process. In some implementations, the freeze-dried reaction components can be stable at room temperature, which can contribute to facilitate handling procedures. In other implementations, the freeze-dried reaction components can be stable around 4° C., or below 4° C. In some implementations, the cartridge 22 can be kept frozen until use. When stability requirements are different between given reaction components included in the FDCF reaction compartment and the purification compartment, a first cartridge that includes the reaction compartment can be provided and stored under a first set of stability conditions, and a second cartridge that includes the purification compartment can be provided and stored under a second set of stability conditions.

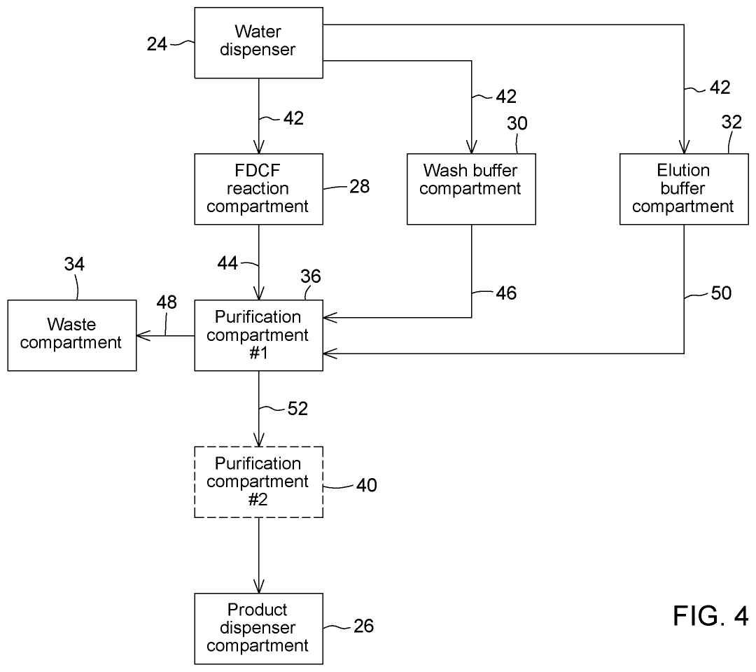

FIG. 4 illustrates an example of fluid communication paths between the compartments of the cartridge. As mentioned above, prior to initiation of the automated process, the compartments can be sealed. Upon initiation of the automated process, for instance when the user closes the top wall 16 of the portable device, and subsequently optionally presses an actuator, fluid communication between the reaction compartment 28 and the water dispenser 24 is established to supply water 42 to the reaction compartment 28 to rehydrate and activate the cell-free reaction components. Rehydration of the cell-free reaction components produces a raw biomolecular products mixture 44 which will include the target biomolecular product after addition of input molecules, such as DNA or cofactors, and incubation at a certain temperature.

In addition, fluid communication can be established between the wash buffer compartment 30 and the water dispenser 24 to supply water 42 to the wash buffer compartment 30 and rehydrate the wash components to produce a washing buffer 46. Fluid communication can also be established between the elution buffer compartment 32 and the water dispenser 24 to supply water 42 to the elution buffer compartment 32 and rehydrate the elution components to produce an elution buffer 50.

Still in accordance with the automated process, once it is determined that the production of the target biomolecular production within the reaction compartment 28 is completed, fluid communication between the reaction compartment 28 and the purification compartment 36 can be established to transfer the raw biomolecular products mixture 44 to the purification compartment 36. Fluid communication can also be established between the wash buffer compartment 30 and the purification compartment 36 to transfer the washing buffer 46 to the purification compartment 36. The purification compartment 36 is thus configured to receive the raw biomolecular products mixture 44 and the washing buffer 46 to allow a washing step to occur within the purification compartment 36, which produces a waste mixture 48 and a washed biomolecular product mixture. The waste mixture 48 can then be transferred to the waste compartment 34 or to an exit port.

Once the washing step is completed, fluid communication can be established between the elution buffer compartment 32 and the purification compartment 36 to transfer the elution buffer 50 to the purification compartment 36. The purification compartment 36 is thus also configured to receive the elution buffer 50 to allow an elution step to occur within the purification compartment 36 when contacting the washed biomolecular product mixture with the elution buffer to produce a purified target biomolecular product 52.

Optionally, a second purification compartment 40 can be provided to receive the purified target biomolecular product 52 for further purification. Further purification can include an additional elution step.

Alternatively, two purification steps can be combined within the same purification compartment, for instance by packing sequentially two or more types of beads within a single purification compartment. Thus, a second purification step may be performed within the first compartment without having to provide a physically distinct additional purification compartment.

In some implementations, the cartridge 22 may be provided with a removable sticker, which can include information relative to its contents and instructions for use, and the sticker may later be applied to a syringe barrel/vial that will eventually contain a dose of the biomolecular product that has been produced. The cartridge 22 can also include a barcode or a radio-frequency identification (RFID) tag that provides manufacturing details, such as volumes of reaction components and water required for production of the target biomolecular product, temperature of the reactions, duration of the reactions, and information about the molecular reporter used.

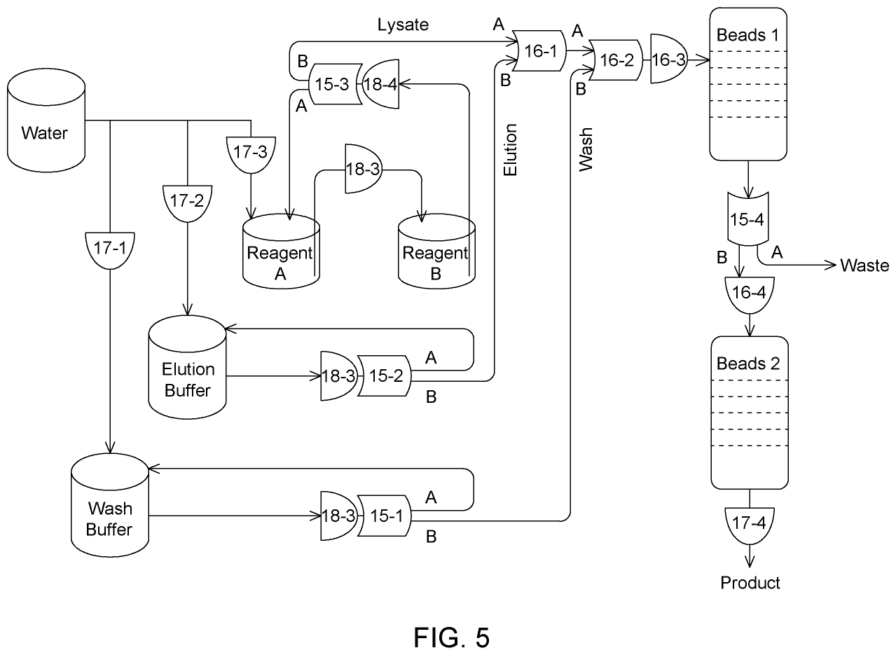

With reference to FIG. 5, a flow chart of an automated process for producing a biomolecular product according to an implementation is shown. In this implementation, pump 17-3 transfers water to the compartment containing reagent A to rehydrate reaction components contained therein. Reagent A can contain reaction components to synthesize the target biomolecular product. This rehydrated solution is then pumped into another compartment containing reagent B by pump 18-3. Reagent B can contain a measurable molecular reporter. Pump 18-4 then transfers the raw biomolecular products mixture comprising the target biomolecular product and the measurable molecular reporter back into the reaction compartment through valve 15-3A or into a first purification compartment (beads 1), which can also be referred to as a purification column, through valve 15-3B, 16-1A, and 16-2, assisted by pump 16-3. Once in the purification compartment (beads 1), the target biomolecular product adheres to the affinity matrix via an affinity tag. Valve 15-4A is opened and non-adhered molecules (waste) flows to the waste compartment.

In accordance with the automated process shown, pump 17-1 transfers water to the wash buffer compartment to rehydrate the wash buffer. Valves 15-1B and 16-2 open and pumps 18-3 and 16-3 transfer the rehydrated wash buffer to the first purification compartment containing beads 1. The wash buffer thus conducts a washing step of the adhered target biomolecular product. The wash buffer and waste washed off the bound target biomolecular product flows to waste through valve 15-4A. Pump 18-3 can also transfer the wash buffer back into the wash buffer compartment through valve 15-1A. Pump 17-2 transfers water to the elution buffer compartment to rehydrate the elution buffer. Valves 15-2B, 16-1A, and 16-2 open and pumps 18-3 and 16-3 transfer the elution buffer to the first purification compartment (beads 1). Valve 15-4B is open and the eluted target biomolecular product is transferred to a second purification compartment (beads 2) by pump 16-4. The eluted product is then transferred by pump 17-4 to a product dispenser compartment. Pump 18-3 can also transfer the elution buffer back into the elution buffer compartment through valve 15-2A.

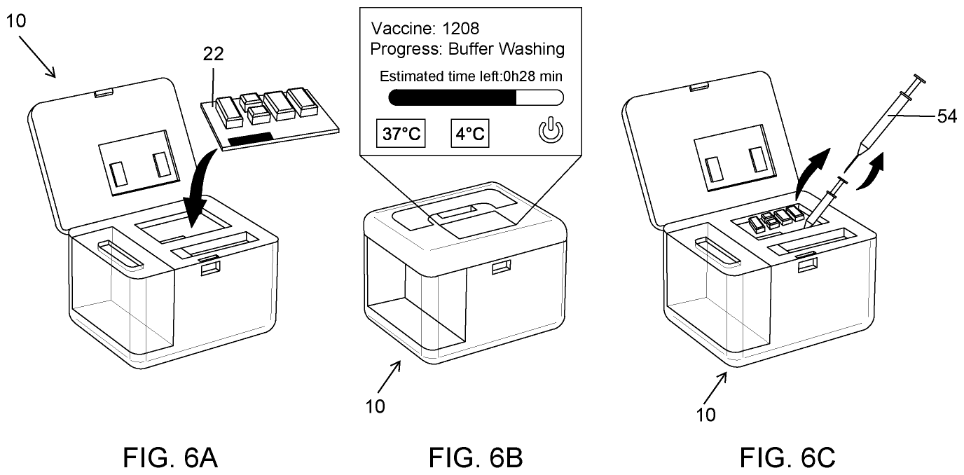

With reference to FIG. 6A to 6C, a portable device 10 for use with a cartridge 22 is shown. In this implementation, the cartridge 22 contains required reagents to produce and purify the target biomolecular product. Firstly, the cartridge 22 is inserted in the portable device 10 (FIG. 6A). The device processor runs an automatic synthesis and purification of the target biomolecular product (FIG. 6B). The purified target biomolecular product is then removed from the portable device 10 and automatically incorporated to an appropriate formulation for delivery, such as within a syringe 54 (FIG. 6C).

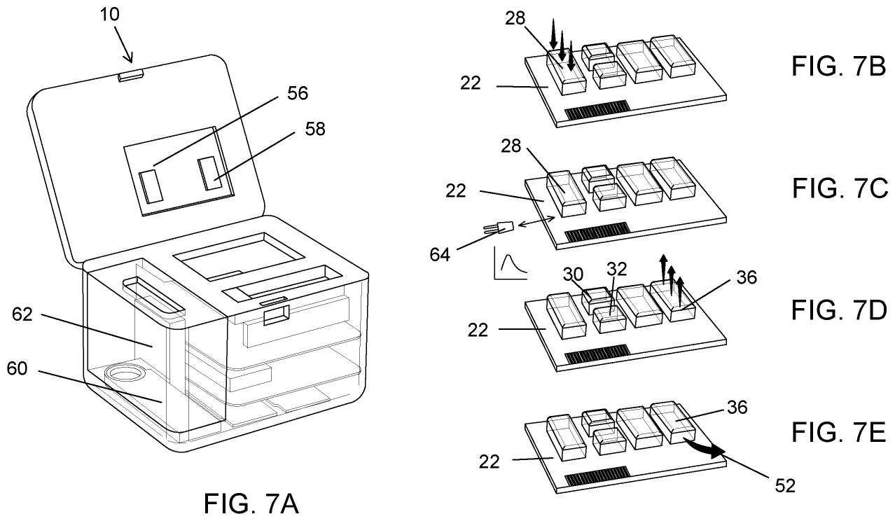

Referring now to FIG. 7A, the internal hardware of device 10 is shown with a heating system 56 and a cooling system 58 that are controlled by the processor 60. The processor 60 controls various operating parameters of the process through a PID Controller 62, such as the temperature and the pressure at which the process is conducted, and the flow of reagents.

FIGS. 7B to 7E illustrate processing steps that can occur within the cartridge 22. FIG. 7B illustrates a freeze-dried cell-free reaction reagents being rehydrated (arrows) in the reaction compartment 28 and heated to a temperature of about 37° C. FIG. 7C illustrates a monitoring of the measurable molecular reporter in the reaction compartment 28 via optical tracker 64. FIG. 7D shows the purification compartment 36 being cooled to about 4° C. as the raw biomolecular products mixture is purified via affinity binding. The wash buffer and elution buffer are rehydrated and wash and elution steps occur sequentially via successive transfer of the wash buffer and elution buffer from wash buffer compartment 30 and elution buffer compartment 32, respectively. Waste is collected in waste compartment 34. FIG. 7E shows a purified target biomolecular product 52 in the purification compartment ready to be transferred to a product dispensing compartment, such as a syringe.

### Cell-Free (FDCF) Reaction Compartment

Cell-free systems can be used as a platform for diagnostic applications and for manufacturing of protein-based products such as therapeutics and lab reagents through in vitro transcription and translation technologies.

In the following paragraphs, the expression “cell-free” can refer to a FDCF, frozen or liquid cell-free system, as it includes shared processes among all three types of cell-free systems. It is to be noted that when the cell-free system is a liquid cell-free system, rehydration of certain components can be omitted.

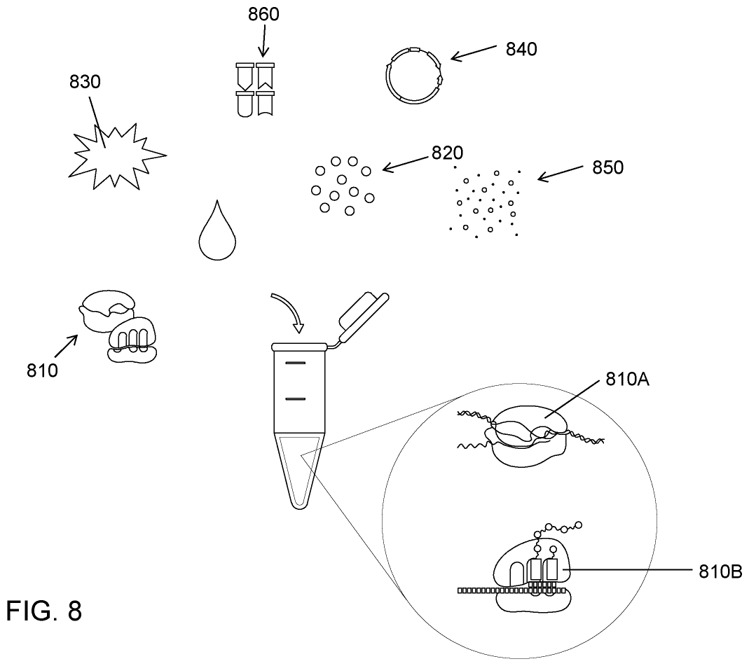

The reaction compartment 28 is configured to include a cell-free system designed to produce a target biomolecular protein. With reference to FIG. 8, cell-free systems contain molecular machinery 810 required to conduct transcription 810A and translation 810B reactions in vitro, and can include or be supplemented with components such as amino acids 820 and an energy source 830. The energy source 830 can be 3-PGA. Cell-free systems can produce the target biomolecular protein outside of a cell directly from the DNA coding sequence, RNA or a combination thereof. This technique can advantageously contribute to overcome some of the limitations that can be associated with traditional cell-based techniques, such as toxicity. Cell-free systems also allow for the direct addition of linear or circular DNA constructs, thereby eliminating the need for cell transformation, selective markers and cell-based cloning steps. In addition, the lack of live cells in cell-free systems can be beneficial in field applications since these systems are biosafe, which in turn can facilitate decentralized use and increased access to therapeutics and diagnostic products even in remote regions when accessibility can be a challenge. For example, referring to FIG. 11, DNA constructs 1100 and crude cell extracts 1110 go through a process of freeze-drying 1120 to create freeze dried DNA constructs 1130 and freeze dried cell extract 1140. Freeze-drying allows for room temperature storage and deployment 1150. A portable device 1160 according to the present invention provides for automated rehydration and purification to create the target biomolecular product 1170. Examples of cell-free systems include crude extract-based methods and recombinant cell-free systems such as the Protein synthesis Using Recombinant Elements (PURE) technology.

Referring back to FIG. 8, cell extract-based cell-free systems utilize a cell lysate to carry out protein synthesis. In this technique, cells grown in cultures are lysed to extract the molecular machinery contained in the cells. The cell extract is then combined with essential components and protein-coding DNA sequences 840 to synthesize proteins. This method has been adapted to create lysates from different host cell organisms (E. coli, CHO, etc.). E. coli S30 based cell-free systems are the most commonly used method due to their high protein yield and cost-effective production. Alternatively, eukaryotic cell extracts, such as insect cell extract, can be utilized to carry on post-translational modifications. Furthermore, extracts can be supplemented with additives 850, such as disulfide bond enhancers, which favour these modifications. Cell extract-based methods are often chosen for manufacturing proteins in vitro, thanks to higher yields produced.

The PURE technology is another option for cell-free protein production. In some PURE technology systems, proteins required to perform protein synthesis are recombinantly cloned with a His-tag and expressed in a host organism for individual purification. Purified fractions are pooled to create a protein solution and combined with a chemical solution containing accessory reagents, such as amino acids 820, an energy source 830, and ribonucleotides 860, that are required to carry out transcription 810A and translation 810B reactions. Although reported protein yields can be lower compared to extract based methods, the PURE technology has the advantage of creating a more controlled environment.

In the context of the present description, the cell-free system contained in the reaction compartment 28 can be any type of cell-free system that is suitable for production of the target biomolecular product. The cell-free system can include a DNA or RNA construct coding for the protein of interest. In some implementations, the DNA or RNA added to the cell-free system includes a sequence, which can be a coding sequence, to produce a given molecular reporter. The molecular reporter can be part of the same expression construct or expressed from a different expression construct. In some implementations, the molecular reporter can be fused to the biomolecule of interest being made. The molecular reporter can be for instance colorimetric, fluorescent, enzymatic, affinity-based, electrochemical, etc. The molecular reporter can be a complete protein, a split protein where only a portion of the protein is expressed in the reaction mix supplemented with the rest of the protein, or be activated using a cofactor. In some implementations, the molecular reporter can be fused to another biomolecule than the protein of interest. In some implementations, the molecular reporter can be the E. coli gene LacZ, encoding for β-galactosidase. The molecular reporter is produced concomitantly with the target biomolecular product, and monitoring of a measurable signal produced by the molecular reported allows tracking of the production of the target biomolecular product. In some implementations, the molecular reporter can be a short peptide sequence, such as ReAsH, which can provide live or endpoint reporting of the amount of protein made or a proxy thereof.

The DNA or RNA constructs of the cell-free system can also include coding sequences for fusion tags, e.g., affinity tags. Examples of affinity tags can include polyhistidine tags (His-Tag), calmodulin tags, cellulose binding domains, chitin-binding domains, maltose-binding domains, glutathione S-transferases, spy-catcher or spy tag. Each specific affinity tag is configured to bind strongly to a particular affinity matrix. These affinity matrices can be provided as beads, which can provide for a large surface area.

In some implementations, the DNA or RNA construct can code for a specific amino-acid sequence that can later on, during purification steps, be used as a cleavage site when a given protease is used in the elution buffer. The cleavage site is generally provided between the protein of interest, i.e., the target biomolecular product, and the affinity tag. Inclusion of such a cleavage site can allow for the removal of the affinity tag from the protein of interest following binding of the affinity tag to the affinity matrix or following in-solution cleavage. The removal of an affinity tag from a protein of interest is generally considered an important step in obtaining clinical grade protein-based therapeutics. As such, affinity tags are often used in conjunction with site-specific proteases which make tag removal possible. With reference to FIG. 13, an example of protease that can be used to cleave an affinity tag from a protein of interest is a protease from the tobacco etch virus, i.e., the TEV protease, which recognizes the amino-acid sequence Glu-Asn-Leu-Tyr-Phe-Gln-(X) and cleaves between the Gln and X residues, where X=Gly or Ser are generally preferred).

In some implementations, the DNA or RNA construct can code for a protein sequence, such as an intein that produces a self-cleaving element, combined with an affinity tag that allows for the purification of a recombinant protein without the need for the use of an external protease. The intein self-cleavage reaction can occur following the addition of a self-cleaving initiator such as dithiothreitol (DTT), 2-mercaptoethanol, cysteine, or hydroxylamine, a change in pH or temperature, thereby releasing the protein of interest from the affinity matrix, e.g., chitin beads or resin. Intein-based cleavage may also be initiated using a change in pH or temperature.

The cell-free system of the reaction compartment 28 can also include additives such as dimethyl sulfoxide (DMSO), trimethylamine N-oxide (TMNO), trimethylglycine and/or inulin. In some implementations, such additives can increase protein yield in the cell-free system.

In some implementations, the cell-free system contained in the reaction compartment 28 are freeze-dried, which can allow the cartridge 22 to be transported to the point-of-care or a remote lab, and rehydrated on site to produce a biomolecular product of interest.

As mentioned above, rehydration of the cell-free system can occur once the cartridge is placed onto the cartridge-receiving section 20 of the portable device 10, and following closure of the top wall 16, and optionally upon activation of an actuator. Water can be supplied via a water dispenser integrated into the portable device. In some implementations, the reaction compartment 28 is incubated at a temperature allowing the protein synthesis reactions to occur, which can range for instance from about 10° C. to about 40° C., as required. Upon rehydration of the cell-free system, which either contains DNA or RNA coding for the target biomolecular product or is supplemented with such components at the start or during the reaction, the processes of transcription and/or translation begin, which leads to protein production. The manufacturing progress can be tracked over time by monitoring a colorimetric, fluorescent, enzymatic, affinity-based, electrochemical, or any other type of molecular reporter (e.g., LacZ, 570 nm or fluorescent protein) expressed at a low rate in the same reaction as the therapeutic, in a parallel co-reaction, or fused to the biomolecule of interest.

In some implementations, reporter tracking can also eliminate the need for a UV monitoring system that is commonly employed in current protein purification process. Alternatively, the cartridge 22 can have portions that are comprised of UV transmitting materials such as quartz, polymethyl methacrylate (PM MA) or cyclic olefin copolymer (COC) to monitor and quantify the protein production via a UV tracker.

Upon reaching a given level of reporter signal for production, which can be previously determined during product development, the raw molecular products mixture 44 is transferred to the purification compartment 36.

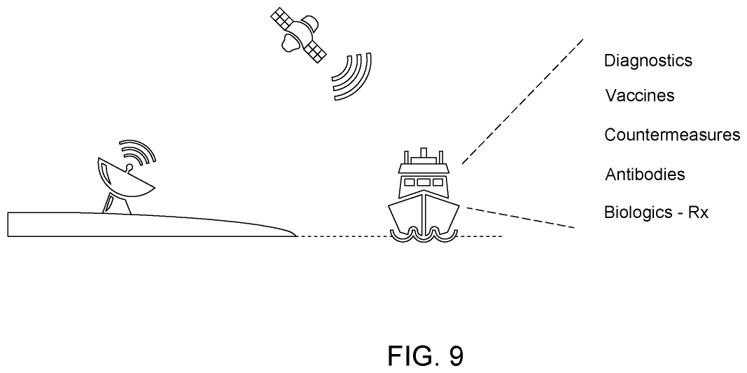

In some implementations, the portable device 10 can be integrated with an on-site DNA synthesizer. The DNA or RNA that can be used for running the cell-free reaction can be produced on-site using the on-site DNA synthesizer and transferred to the reaction compartment 28 providing instructions to make any desired molecules. This type of implementation can enable to go directly from digital code to specific molecules programmed by the code. FIG. 9 illustrates an electronic transfer of DNA sequences encoding instructions for manufacture of a target biomolecular product. This electronic transfer of information would allow ad hoc therapeutics to be synthesized in remote locations.

### Purification Compartment

The purification compartment 36 is configured to include purification components that are active or activable upon rehydration or washing. The purification components can be provided as dried purification components, for instance as freeze-dried purification components. This is contrast with conventional methods, where purification components, such as purification beads, are traditionally supplied in a water/ethanol suspension that needs to be kept refrigerated. Providing purification components as dried purification components can provide increased stability at room temperature and be beneficial for their shipping and storage.

The purification components include an affinity matrix configured to bind to the target biomolecular product contained in the raw biomolecular products mixture 44. The binding of the target biomolecular product to the affinity matrix can occur directly onto the matrix surface or indirectly via capturing with another protein/peptide/biomolecule that can be immobilized on the matrix surface. In general, any affinity chromatography, which is a liquid chromatographic technique that takes advantage of biological interactions for separating specific analytes in a sample through their binding to immobilized substrates or inhibitors, or solvent extraction technique can be applied to the purification scheme described herein.

Affinity chromatography uses interaction between components, e.g., a ligand and an enzyme, to separate molecules of interest from a liquid. By immobilizing one component of the system on an insoluble porous support, the molecule of interest can be selectively adsorbed. Then, once impurities have been washed away, the molecule of interest can be eluted through any procedure that results in dissociation of the complex. Many immobilized components are available, which can be used in conjunction with a wide variety of affinity tags including polyhistidine tags, cellulose-binding domains, chitin-binding domains, maltose-binding domains, glutathione S-transferases and Spy tag and FLAG tag.

Binding of the target biomolecular product to the affinity matrix can be conducted at various temperatures, which can depend on the target biomolecular product itself and/or on the type of affinity matrix used. For instance, in some implementations, binding can be conducted at room temperature, while in other implementations, binding can be conducted at a temperature within a range of about 2° C. to about 10° C. The purification scheme described herein can be adaptable to a plurality of protein purification matrices. Examples of affinity matrix can include nickel-nitrilotriacetic acid (Ni-NTA) beads, protein-A beads, chitin-binding beads, amylose resin, agarose or cellulose matrices, or any other types of beads or surfaces modified with other molecular components such as proteins, peptides, or chemicals that are suitable to capture the target biomolecular product.

A protease such as that of the tobacco etch virus, i.e., TEV protease, can be used as a method for cleavage and purification of fusion proteins. The sequence for the protease cleavage site, such as the TEV cleavage site, is placed between the protein of interest and an affinity tag. Once the fusion construct is expressed, it can be bound to the affinity matrix using the affinity tag. The protein of interest can then be cleaved off of the affinity matrix, leaving the affinity tag on the beads. The TEV protease has demonstrated efficiency at low temperatures, e.g., at 4° C., allowing cleavage while minimizing non-specific proteolytic degradation of target proteins. Other commonly available proteases include factor Xa, thrombin, enterokinase and human rhinovirus 3C protease.

In some implementations, the protein of interest can be fused to a histidine tag (His tag) and includes a TEV cleavage site at the N-terminus. The His tag is then bound to a modified surface designated for capture, such as Nickel-NTA beads, chitin beads, or beads modified with antibodies, or proteins or peptides capable of reversible or irreversible conjugation to affinity partners fused to the protein of interest. Once washing steps are completed, the protein of interest is cleaved with a TEV protease. The TEV protease can be fused to a chitin-binding domain, allowing for its removal post cleavage through a compatible affinity matrix, such as chitin beads.

In some implementations, when a cleavage site is included in the sequence coding for the protein of interest to subsequently allow cleavage of the protein of interest from its affinity tag, a second purification compartment 40 can be included in the cartridge 22 to bind the protease and/or the cleaved affinity tag. The second purification compartment 40 can thus include a second affinity matrix that is different from the affinity matrix used in the first purification compartment 36, depending on the affinity tag being captured.

For instance, in one scenario, a Chitin-binding/GST-tag/TEV Protease (S219V) chimera can be used for cleavage of an affinity tag from the recombinant proteins and for later capture of the TEV protease. The recombinant proteins are expressed in the cell-free environment using a His-tag/TEV-cleavage-site/Construct syntax. Once the expression is completed, these precursor constructs are bound to Nickel/Cobalt NTA beads. The TEV chimera is then added to cleave the desired construct free of the His tag. TEV protease can then be removed by passing the cleaved proteins through an affinity matrix containing either chitin beads or glutathione beads to capture the cleaving enzyme, in this case the TEV protease, as the eluted protein of interest passes through the second affinity matrix. Although a single affinity tag can be used, it may be advantageous to use a GST tag for initial purification of TEV protease, and a chitin tag for secondary capture of the TEV protease. The TEV construct needs to be expressed and purified prior to use in this scheme. Histidine tag would not be suitable for its purification since it would cause binding to the Nickel-NTA beads during on-column cleavage portrayed above and thus would reduce efficiency cleavage. Moreover, while chitin binding domain is good at binding, it does not easily lend itself to elution for purification purposes. As such, a GST tag was used for purifying TEV.

This example scenario is illustrated in FIG. 13, where a TEV protease coupled to a chitin-binding domain and having a glutathione S-transferase (GST) tag can be used to cleave the protein of interest from its 6×His affinity tag. It is to be noted that various other tags can also be used to apply a similar purification scheme. An untagged protein of interest, or untagged target biomolecular product, can be advantageous for use as a therapeutic product.

Other binding techniques/cleavage techniques may also be utilized. For instance, TEV cleavage site and TEV protease can be replaced with another cleavage site and corresponding protease (e.g. factor Xa). The protein of interest may also be expressed using affinity tags other than His tag. TEV protease used for cleavage can be histidine-tagged and removed through incubation with Ni-NTA beads, or can be tagged with another affinity tag or a combination thereof. A desalting compartment may be used before, after or in between any affinity compartments.

In summary, the integrated molecular purification scheme described herein can be configured to follow these steps:
Binding a protein of interest that can be tagged with an affinity tag to an affinity matrix such as Ni-NTA, chitin beads or beads/surfaces modified with peptides/proteins or chemicals enabling binding to tagged or untagged protein of interest; and
Release of the product using either an enzymatic cleavage component such as TEV protease, a self-cleaving initiator purification component, or a chemical purification component configured to compete with the affinity tag for the binding sites, such as imidazole.
As mentioned above, when another protein such as TEV protease is used, capturing of this protein using a second affinity matrix to obtain a final purified product may be beneficial.

### Wash Buffer Compartment

The wash buffer compartment 30 is configured to include wash components that can eventually be supplied to the purification compartment 36. The wash components can be provided as dried, for instance freeze-dried, wash components within the wash buffer compartment 30 and produce, upon rehydration, a washing buffer 46 that can remove non-specific impurities from the raw biomolecular products mixture. The washing buffer thus removes the non-target biomolecules by disturbing their weak or non-specific interaction with the affinity matrix, while the target biomolecular product remains bound to the affinity matrix via its affinity tag or through other binding mechanisms. Wash components can include for instance salts, and detergents. In some implementations, the wash buffer can contain 20 mM Imidazole, 500 mM NaCl, 50 mM Trizma base pH 7.5 and 5% glycerol. The wash buffer may or may not contain a reducing agent such as DTT or TCEP depending on the application.

### Elution Buffer Compartment

The elution buffer compartment 32 is configured to include elution components that can eventually be supplied to the purification compartment 36. The elution components can be provided as dried, for instance freeze-dried, elution components within the elution buffer compartment 32 and produce, upon rehydration, an elution buffer 50. The elution buffer 50 can be introduced into the purification compartment 36 once the washing is completed, at a volume that can be based on the target concentration of the target biomolecular product and the optical reporter measurements. The elution buffer 50 can have different roles. In some implementations, the elution components of the elution buffer 50 can include a chemical agent that competes with the affinity tag of the protein of interest for the binding sites of the affinity matrix, which elutes the protein of interest off of the affinity matrix. An example of such chemical agent is imidazole or histidine when used with Ni-NTA beads. In other implementations, the elution components of the elution buffer 50 can include an enzyme/protein capable of releasing the protein of interest off of the affinity matrix. An example of such enzyme is the TEV protease. As mentioned above, when the TEV protease is used, a second affinity matrix can be used to capture the TEV protease and further purify the target biomolecular product. In yet other implementations, the elution components of the elution buffer 50 can include a compound that is capable of triggering self-cleavage of the protein of interest that is bound to the affinity matrix, thereby allowing the release of the protein of interest from the affinity matrix. In some implementations, the components of the elution compartment, which may or may not include a protease, can be supplied as dried or freeze-dried components.

The elution of the target bimolecular product produces a purified target biomolecular product 52, the user can retrieve a dose of the purified target bimolecular product 52 for immediate usage or, in the event that the dose of purified target biomolecular product 52 is not required immediately and/or some transportation is required, the portable device can hold the produced dose at 4° C. until needed. The dose can be retrieved and stored in a syringe 54 or a vial, as shown in FIG. 6C.

In some implementations, the device can be configured to record parameters and measurements of volumes and concentrations associated with the produced dose of the purified target biomolecular product 52. The device can also be configured to notify the user that the dose is ready to be retrieved either audibly or visually via the indicator screen. Once the production of the purified target biomolecular product is completed, the top wall of the device can be opened, the cartridge can be discarded without specialized handling, and the dose of the purified target biomolecular product retrieved and used. It is to be noted that in other implementations, the device can also receive the cartridge 22 in any suitable way other than within the reaction chamber and/or the cartridge 22 can be removed from the device other than by opening the top wall of the device.

Tracking System

In some implementations, the device can include a tracking system to monitor the production of the target biomolecular product via the production of a measurable signal by the molecular reporter. As mentioned above, the molecular reporter can be colorimetric, fluorescent, enzymatic, affinity-based, electrochemical, or any other type of molecular reporter that produces a measurable signal. In some implementations, the molecular reporter produces a measurable signal that can be detected with a spectrophotometer at a given wavelength. For instance, the molecular reporter can be the LacZ reporter gene, and monitoring of the production of the target biomolecular product can include measuring the absorbance at about 570 nm. In other implementations, monitoring of the production of the target biomolecular product can include production of a fluorescent protein such as red fluorescence protein and recording fluorescence emission upon excitation at a specific wavelength.

In some implementations, live monitoring of the manufacturing process can allow to control and optimize the duration of the process for manufacturing the purified target biomolecular product. Upon reaching a given threshold level of the signal produced by the molecular reporter, which can be previously determined during product development, the raw biomolecular products mixture is transferred to the purification compartment to be subjected to purification steps.

Live monitoring of the manufacturing process of the purified target biomolecular product can be advantageous in various scenarios. For example, in the case of an acute emergency such as a snake bite, monitoring of the manufacturing process can enable the portable device to produce a single antivenom dose as soon as possible. An example of an antivenom construct is shown in FIG. 12. The structure includes a TEV site in front of the antivenom construct to remove the 6× His tag from the final antivenom product. Conversely, in scenarios where time is not necessarily critical, such as for the production of a seasonal flu vaccine, production of the vaccine can be programmed to produce a maximum number of doses and manufacturing can be extended until production is saturated, as determined by monitoring the reporter.

Live monitoring of the manufacturing process of the purified target biomolecular product via a molecular reporter can also give rise to opportunities for machine learning to optimize yield and also to determine the relationship between the molecular reporter and the manufacturing of the biomolecular product during either product development, e.g., in a laboratory setting, or during actual manufacturing in the portable device.

In some implementations, live monitoring of the manufacturing process can enable quality metrics to be collected on the manufacturing process. Drug manufacturing requires batch number traceability and time course molecular reporter signal can contribute to generate an information set that can be linked to a manufactured dose of the purified target bimolecular product drug that will be administered to each patient.

As mentioned above, in some implementations, the use of a molecular reporter to monitor the production of the purified target biomolecular product can also eliminate the need for a UV monitoring system that is commonly employed in conventional protein purification processes.

### Temperature Control Systems

The portable device can include a temperature control system to regulate the temperature within certain compartments of the cartridge.

In some implementations, the temperature control system includes a heating system configured to provide local heating to the cell-free reaction compartment during the production of the raw biomolecular products mixture, i.e., as the protein expression reactions occur. Heating may also be applied during purification, for instance to increase the rate at which a protease cleaves products off of the affinity matrix. The heat provided can be pre-determined according to previously conducted validation protocols, and can be monitored via a temperature sensor located in proximity of the reaction compartment. For example, in some implementations, heat provided to the reaction compartment can be such that the protein expression reactions can occur within a range of between about 16° C. and about 37° C. In some implementations, the heating system can be omitted.

In some implementations, the temperature control system includes a cooling system configured to provide local cooling to the expression, purification or buffer compartments. In some implementations, cooling is only applied to the purification chamber. The cooling of the purification compartment can be such that is it maintained within a temperature range of about 2° C. to about 10° C. In some implementations, the temperature range can be determined according to stability requirements, and/or according to previous validation protocols determining desired operating conditions for the washing and elution steps. In some implementations, the cooling system can also cool the product dispenser compartment to provide enhanced stability conditions to the purified target biomolecular product prior to being administered to a patient.

### Tracing System

An aspect to consider in the production of protein-based therapeutics is the traceability of a production batch, such that if there are adverse effects experienced by patients to whom that specific production batch was administered, public health officials can trace back and contact other patients that also received that specific production batch.

In some implementations, the portable device described herein can be configured to assign a unique identifier to a given production batch and link manufacturing parameters to the given production batch using a block chain-based open ledger or using traditional databases.

For a given batch of the purified target biomolecular product produced, the portable device can be configured to collect a series of manufacturing metrics, including the date of production, the geographical location of manufacturing, the reaction duration, the reaction temperature, the origin of the cartridge, the DNA/RNA sequence used, the production yield, pressure and/or flow rates, optical intensity from the optical trackers, and/or any other suitable reaction quality metrics, which can be stored in an immutable block chain ledger or traditional database. This data set will be used to comply with regulatory requirements, and can also enable meta-level analysis of product improvement and performance monitoring. Data can be stored to servers accessed via the internet or cellular signal, or stored on the device until network connection.

In some implementations, the collection of such data can provide an opportunity for the application of machine learning across the system through a global network of users. In addition, the portable device can provide scalability benefits for performing nonlinear deep neural networks and variational inference to impute the expressions of DNA/RNA construct in the cell-free system.

In some implementations, a web authentication of the cartridge to be used in the portable device can be used, allowing for tracking and identification of the batch of purified target biomolecular product produced through third-party servers or block chain.

### Processor

As mentioned above, the portable device is designed and constructed such that the production of the purified target biomolecular product is conducted according to an automated process. In some implementations, the portable device includes a processor that is operatively connected to various hardware components, and to an electronic circuitry that connects the hardware components that include the fluidics components together. The processor can thus be operatively connected to the actuator if present, and to one or more of the fluidic system, the heating system, the cooling system, the optical tracker, and the tracing system. In some implementations, the processor can also be operatively connected to a drug delivery integration system. The processor is configured to receive signals from the respective hardware components, and to generate corresponding signals to control the automated production process.

In some implementations, the operation of the processor is performed according to a software that can provide a user interface, and that can allow automated control of the manufacturing steps for the production of the purified target biomolecular product. The software may be unique to each cartridge or can be operated by the user at the point of production to adjust the reaction and purification parameters. Furthermore, the software can allow for more data capture in the process of synthesis and/or purification of the target biomolecular product. For example, by analyzing the data from the pressure and flow sensors in the fluidic system, the processor can adjust the flow rates and timing of the pumps to ensure that synthesis and/or purification of the target biomolecular product can run to completion, even if there is a partial blockage in a fluidic communication line. Monitoring the pressure sensor(s), the flow sensor(s), the temperature sensor(s) and the optical trackers also provides metrics to the software to help determine whether the target biomolecular product was created within lab validated production protocols. Furthermore, through optical monitoring of the measurable molecular reporter produced concomitantly with the target biomolecular product, the processor can transfer the raw biomolecular product mixture to the purification compartment when synthesis plateaus, for instance. Transferring the raw biomolecular products mixture when synthesis plateaus, as opposed to a predetermined time period or when a product threshold is met, can contribute to decrease the overall production time.

### Drug Delivery Loading and Preparation

In some implementations, the biomolecular product or the combination of biomolecular products generated using the device can be automatically prepared for therapeutic applications. For example, the biomolecular product can be loaded into liposomes or combined with any other reagent used for drug delivery. The formulation components can be made within the device or provided as part of the cartridge. The formulation components can be provided in solution, in dried, liquid, frozen or freeze-dried formats.

### Alternative Implementation of the Portable Device

With reference to FIGS. 14A to 14D, 15A, 15B and 16, an alternative implementation of the portable device is shown. In this implementation, the housing of portable device 110 includes with a bottom wall 112, side walls 114, and a top wall 116. The side walls 114 and top wall 116 together define a reaction chamber 118. As shown in FIG. 15, a processor 160 may be operatively connected to a touchscreen 162 to facilitate an initiation of the automated process as well as track metrics of the synthesis and/or purification process. The processor 160 is operatively connected to electronic circuitry 164 that operates the pumps and valves.

In the implementation shown, the reaction chamber 118 contains various compartments used for the purification of a target biomolecular product, and a manifold plate 122. In some implementations, the reaction chamber 118 can further include a reaction compartment (not shown). Given purification components involved in the production of a purified biomolecular product are contained in the various compartments, as will be explained in further detail below. When a reaction compartment is present, reaction components can be contained in the reaction compartment, or the reaction compartment can be supplied with a raw biomolecular products mixture such as a lysate.

The manifold plate 122 includes fluidic channels 1600 that fluidly connect selected compartments together. In some implementations, the fluidic channels 1600 of the manifold plate 122 can contribute to shortening the distance between the compartments and can facilitate reducing dead volume within the reaction chamber. The manifold plate 122 can also simplify the assembly of the portable device, which can improve scalability and lower overall cost of manufacturing the portable device.

Alternatively, the manifold plate 122 can be omitted, and the fluidic channels can be made of tubing, for instance.

In some implementations, the compartments can be as shown in FIGS. 10A to 10C, or alternatively, the compartments can be substantially tubular. It is to be understood that any size and shape of compartments that can serve as a receptacle for containing the desired reagents and receiving a liquid therein, and that can fit within the reaction chamber, can be suitable.

The components contained in the compartments are moved through the fluidic channels 1600 via a fluidic system, which can include and one or more pumps 1610 and associated valves 1620. The fluidic system can optionally include a pressure and/or a flow sensor. In some implementations, the fluidic channels 1600 can be etched, carved, embossed or molded into the manifold plate 122. When the fluidic channels 1600 are etched, an etching machine such as a CNC machine can be used to provide the fluidic channels 1600. In some implementations, the manifold plate 122 can be made of two etched acrylic sheets can be coupled together to create the fluidic channels 1600 that connect the compartments together, although other types of materials can also be used. In this implementation, the two acrylic sheets can be coupled together with a pressure sensitive adhesive, such as with Adhesives Research's ARseal 90880™, which is a polypropylene film coated on both sides with an inert silicone adhesive. In other implementations, the two etched sheets, made of acrylic or another material, can be bound using a pressure controlled lamination process and/or a temperature controlled lamination.

In some implementations, at least one of the compartments can be removably coupled, or removably engaged, with the manifold plate 122. In order to do so, the manifold plate 122 can further comprise compartment-coupling portions that enable coupling with a corresponding one of the compartments. In such implementations, the compartment-coupling portions can be provided in a compartments-receiving section 120 of the manifold plate 122. Alternatively, the compartment-coupling portions can be integral with the manifold plate 122. In the implementation shown in FIG. 16, a binding buffer compartment 124, a wash buffer compartment 130, an elution buffer compartment 132, as waste buffer compartment 134 and a product dispenser compartment 126 are each coupled to a corresponding compartment-coupling portion of a compartments-receiving section 120 of the manifold plate 122.

To enable comprehensive tracking of the movement of the various liquids that include components of interest to produce the biomolecular product in the fluidic channels 1600 using optical trackers (e.g., fluorescent or color) or camera-based monitoring, the fluidic channels 1600 can be etched into clear acrylic sheets, such as poly (methyl methacrylate) (PMMA). An advantage of PMMA is that it is UV transmitting (transparent to UV light), biocompatible, and compatible with CNC machines for ease of manufacturing. In some implementations, an optical tracker in optical communication with the fluidic channels can be used for monitoring fluid circulation within the fluidic channels.

In some implementations, the raw target biomolecule product may be synthesized within the portable device 110 in a reaction compartment as described above, or can be synthesized independently in any cell-free system. When synthesized independently, the raw target biomolecular mixture can be introduced into the reaction compartment of the portable device 110 or through a reaction inlet 128 contained in the manifold plate 122. The reaction inlet 128 can be an adapter, such as adapter 1800 shown in FIG. 18. The raw target biomolecule product is then purified in the purification compartment 136 with purification components 1630, such as an affinity matrix configured to bind to a target biomolecular product contained in the raw target biomolecular mixture, as described above. Other components used to purify the raw target biomolecular mixture can include a binding buffer contained in a binding buffer compartment 124, a wash buffer contained in a wash buffer compartment 130, and an elution buffer contained in an elution buffer compartment 132. The device 110 can also include a waste compartment 134.

In some implementations, the purification compartment 136 can be a purification column with an inlet 1640 enabling fluid communication with the binding buffer compartment 124, the elution buffer compartment 130, and/or the elution buffer compartment 132, and an outlet 1650 enabling fluid communication with the waste compartment 134, an exit port, and/or a product dispenser compartment. Inlets 1640 and 1650 are connected to fluidic channels 1600A and 1600B, respectively, via two purification ports 1660. The purification ports 1660 can be an adapter, such as adapter 1800 as shown in FIG. 18, configured to connect with inlet 1640 and outlet 1650. Alternatively, in the implementation shown in FIG. 16, the purification compartment 136 is connected to the purification ports 1660 with tubing 1670.

In the implementation shown in FIG. 16, the binding buffer compartment 124, the wash buffer compartment 130, the elution buffer compartment 132, the waste compartment 134, and a product dispensing compartment 126 containing the purified target biomolecular product connect to the manifold plate 122 via the compartment-coupling portions of the compartments-receiving section 120 of the manifold plate 122. In turn, the compartments 124, 126, 130, 132, and 134 are in fluid communication with the fluidic channels 1600 via fluidic ports, which can be for instance high-pressure compatible fluidic ports. The compartments 124, 126, 130, 132, and 134 can be tubes or vials that have outer threads, e.g., screw top tubes or screw top vials, such that the tubes or vials can be individually removable from the compartments-receiving section 120 of the manifold plate 122 by threading the screw top tubes or screw top vials into the compartments-receiving section 120, i.e., within corresponding compartment-coupling portions. The compartment-receiving section 120 can be a plate having openings extending therethrough defining a corresponding compartment-coupling portion. In some implementations, the openings can be drilled or bored into the compartments-receiving section 120 or the manifold plate 122 with threading that corresponds to threads on the various compartments.

Referring now to FIG. 17, an example of a fluidic port 1700 is shown with threading 1710, an o-ring seal 1720 and an opening 1730 extending therethrough. The opening 1730 of the fluidic port 1700 enables fluid communication with a corresponding fluidic channel 1600. The fluidic port 1700 can be inserted into an opening defined in the manifold plate 122, the opening having a fluidic port engagement surface enabling the fluidic port to be fixedly attached to the manifold plate 122. In some implementations, the fluidic port 1700 can be screwed to the fluidic port engagement surface of the opening, or can be glued thereto. Any type of mechanical attachment can also be suitable, including for instance one or more bolts. Alternatively, the fluidic ports can be integral in the manifold plate 122. In some implementations, when the fluidic ports 1700 are integral with the manifold plate 122, CNC thread milling can be used to manufacture the fluidic ports relatively quickly and inexpensively.

The fluidic ports 1700 are further configured to engage with an adapter 1800. The fluidic port 1700 can be configured to removably connect to an adapter 1800, as shown in FIG. 18. In turn, the adapter 1800 can be configured to receive a feed tube that extends within a corresponding compartment to facilitate transfer of the fluids from one compartment to another through the fluidic channels. In some implementations, the feed tube can have an inner diameter ranging from about 0.2 mm to about 2 mm. A pump 1900, such as the solenoid pump shown in FIG. 19, is mounted to the manifold plate 122 via mounting screws 1910. The pump 1900 allows for the transfer of respective liquids contained in the given compartments through the feed tube into the manifold plate 122.

In this implementation of the portable device, the raw target biomolecular product can be synthesized in a separate device or module using cell-free systems known in the art and/or described herein. Alternatively, as shown in FIGS. 20A and 20B, the raw target biomolecular mixture product can be synthesized within the portable device 210 within a reaction compartment 228 that is coupled, e.g., removably coupled, to the manifold plate 222. The components in the binding buffer compartment 224, the washing buffer compartment 230, the elution buffer compartment 232, the reaction compartment 228, the waste compartment 234, and the product dispensing compartment 226 are in fluid communication with the fluidic channels 2000 in the manifold plate 222 via feed tubes 2030. The components are transferred between the compartments and through the purification compartment 236 via a fluidic system of pumps 2010 and valves 2020. The compartments 224, 226, 228, 230, 232, and 234 can be tubes or vials that can be removable from corresponding compartment-coupling portions of the compartments-receiving section 220 of the manifold plate 222.

In some implementations, such as in the implementation shown in FIGS. 20A and 20B, the purification compartment 236 can be removably attached to the manifold plant 222 via purification ports 2060. The purification compartment 238 can include an inlet 2040 to enable fluid communication between the purification compartment 236, and optionally the binding buffer compartment 224, the wash buffer compartment 230, and/or the elution buffer compartment 232. The purification compartment 236 can further include an outlet 2050 to enable fluid communication between the purification compartment 236 and the waste compartment 134, an exit port, and/or a product dispenser compartment 226. In FIG. 20A, the inlet 2040 and the outlet 2050 are connected to fluidic channels 2000A and 2000B, respectively, via purification ports 2060 and tubing 2070. As shown in FIG. 20B, the purification ports 2060 can extend outwardly from the manifold plate 222, such as from the top of the manifold plate 222. In the implementation shown, the purification ports 2060 include a bend or elbow to align with each other and create a direct path for the purification compartment 236 to extend therebetween.

When the raw target biomolecular mixture product is synthesized within a reaction compartment 228 of the portable device 210, a tracker, such as optical tracker 264, can be located adjacent to, or in proximity of, the reaction compartment 228 to monitor the progression of the synthesis reaction. The optical tracker 264, shown in detail in FIG. 21, is operatively connected to the processor to monitor the protein synthesis reaction by measuring the raw biomolecular products mixture 244 (i.e., a measurable molecular reporter produced concomitantly with the target biomolecular product). In some implementations, the optical tracker 264 can be a fluorescence detector. In the portable device 210, the optical tracker 264 is shown as being attached to the housing 218 holding the manifold plate 222, although other types of connection are also possible.

In the implementation shown in FIG. 21, the optical tracker 264 comprises a photodetector 268, an emission filter 270, an aperture 272, and an excitation light emitting diode (LED) 274. When the reaction compartment 228 is coupled to the manifold plate 222, the lower portion of the reaction compartment 228 that contains the reaction components is adjacent to the aperture 272. In this implementation, the LED 274 is located underneath the reaction compartment 228 to measure the molecular reporter by emitting light from below the reaction compartment 228 and exciting the molecular reporter within the reaction compartment. The emitted fluorescent light is measured by the photodetector 268 after passing through the aperture 272 and the emission filter 270. FIG. 22 shows measurements taken using an optical tracker to detect green fluorescent protein (with and without an emission filter).

FIGS. 23A to 23D illustrate another implementation of an optical tracker 364 with an emission filter 370. The production of the raw biomolecular products mixture 344 is measured via the optical tracker 364. FIG. 24 shows an optical tracker 464 that can measure the measurable molecular reporter simultaneously in two reaction compartments 428.

FIG. 25 illustrates an example of an automated process using a manifold to provide fluid communication paths between the various compartments. In some implementations, a first step can involve loading compartments that include given components into the portable device. Loading the compartments into the portable device can include connecting the compartments to the manifold plate 122. When the raw target biomolecular products mixture 504 is synthesized in the reaction compartment 528, upon initiation of the automated process, such as through a touch screen display 521, the processor 520 initiates the synthesis reaction. In some implementations, once the reaction components are loaded into the portable device, the synthesis reaction can be initiated by the processor 520 controlling the heating and cooling systems 517 to provide a controlled temperature for synthesis reaction.

The reaction compartment 528 can be configured to include a cell-free system designed to produce a target biomolecular protein. When the raw target biomolecular products mixture 504 is synthesized in the reaction compartment 528, the temperature of the synthesis reaction can be monitored by the processor 520 via temperature sensors 518. The processor 520 can be configured to control the heating and cooling systems 517, which can contribute to maintaining reaction conditions according to pre-determined validation protocols. To maintain a substantially homogenous mixture and provide oxygen to enhance the rate of synthesis in the reaction compartment 528, air can be periodically pumped into the reaction compartment 528 during the synthesis reaction. A pump 516 can be activated by the electronic circuitry 519 periodically via a signal from the processor 520. The pump 516 can be activated based on pre-determined intervals and/or feedback from the optical tracker 522, which can be for instance a fluorescent sensor. The optical tracker 522 can be configured to monitor the production of the target biomolecular product and provide feedback to the processor 520. The processor 520 can further adjust the heating and cooling systems 517 and the fluidic system, for instance to maintain reaction conditions (i) until the optical tracker 522 indicates that a desired product threshold is met or (ii) for a pre-determined duration for the synthesis reaction. In some implementations, the synthesis reaction can range for instance from about 4 to about 16 hours.

Alternatively, synthesis of the raw biomolecular products mixture 504 can be completed in a separate device or module. When the raw biomolecular products mixture 504 is synthesized in a separate device, initiation of the automated process, such as via a touchscreen display 521, can establish fluid communication between the binding buffer compartment 524 and the purification compartment 536. When a binding buffer 501 is not used, initiation of the automated process can establish fluid communication between the reaction compartment 528 and the purification compartment 528.

In some implementations, the portable device can include a binding buffer compartment 524 configured to contain binding buffer 501 to equalize the purification components, such as an affinity matrix, before the raw target biomolecular products mixture 504 is transferred to the purification compartment 536. When a binding buffer 501 is used, the processor 520 can signal to the electronic circuitry 519 to activate pump 510 of the fluidic system so as to establish fluid communication between the binding buffer compartment 524 and the waste compartment 534 by passing through the purification compartment 536. Specifically, 3-way valves 507, 508, 509, and 515 are activated to create a fluid connection and then pump 510 is activated to transfer the binding buffer 501 through the purification compartment 536 to the waste compartment 534 or an exit port.

Still in accordance with the automated process, the processor 520 establishes fluid communication between the reaction compartment 528 or reaction inlet and the waste compartment 534 through the purification compartment 536 by signalling motherboard 519 to activate the fluidic system. Specifically, 3-way valves 509 and 515 are activated to create a fluid connection between the reaction compartment 528 and the waste compartment 534, and then the pump 510 is activated to transfer the raw target biomolecular products mixture 504 through the purification compartment 536. Unbound molecules (waste 506) flow through valve 515 to the waste compartment 534 or to an exit port. Specifically, the affinity tags on the raw biomolecular products mixture 504 bind to the affinity matrix in the purification compartment 536. The target biomolecular product is thus retained on the affinity matrix in the purification compartment 536 creating a waste mixture 506 of unbound molecules (waste 506) that flows to the waste compartment 534 or an exit port. In some implementations, a tracker or optical sensor 514 can monitor whether some of the target biomolecular product does not bind to the affinity matrix, thereby resulting in unbound target biomolecular product that undesirably flows from the purification compartment 536 to the waste compartment 534, and the tracker or optical sensor 514 can provide feedback to the processer 520.

Still in accordance with the automated process, once the raw biomolecular products mixture 504 has been transferred through the purification compartment 536, the processor 520 establishes fluid communication between the wash buffer compartment 530 and the waste compartment 534 through the purification compartment 536. The processor 520 signals the electronic circuitry 519 to activate 3-way valves 507, 508, 509, and 515 and pump 510 transfers the wash buffer 502 through the purification compartment 536 and into the waste compartment 534. The transfer of the wash buffer 502 through the purification compartment 536 allows a washing step to occur and a washed biomolecular product mixture is retained on the affinity matrix in the purification compartment 536. In some implementations, a tracker or optical sensor 514 can monitor the amount, such as the volume, of wash buffer 502 that flows from the purification compartment 536 to the waste compartment 534, and provide feedback to the processer 520 accordingly. In turn, such monitoring can enable the processor 520 to control the total amount of washing buffer 501 to wash the bound target biomolecular product. Furthermore, the processor 520 can determine when to communicate to the electronic circuitry 519 to close 3-way valves 507, 508, 509, and/or 515 and/or shut off pump 510 based on data collected by the tracker or optical sensor 514 during monitoring.

Once the washing step is completed, fluid communication between the elution buffer compartment 532 and the product dispenser compartment 526 through the purification compartment 536 is established. The processor 520 signals the electronic circuitry 519 to activate valves 508, 509, and 515 and pump 510 transfers the elution buffer 503 through the purification compartment 536, allowing an elution step to occur within the purification compartment 536. When the washed biomolecular product mixture comes in contact with the elution buffer, the elution buffer 503 causes the target protein to release from the affinity matrix and a purified target biomolecular product 505 flows into the product dispenser compartment 526. The product dispenser compartment 526 can be a removable tube or ready to use syringe barrel/vial. In some implementations, a tracker or optical sensor 514 can monitor the amount of target biomolecular product that flows from the purification compartment 536 to the product dispenser compartment 526 and provide feedback to the processer 520 accordingly. In turn, such monitoring by the processor 520 can enable the processor 520 to control the total amount of elution buffer 503 required to elute the purified target biomolecular product 505 and thus maintain higher concentrations of the target biomolecular product and prevent unnecessary dilution with the elution buffer 503. In some implementations, the processor 520 can determine when to communicate to the electronic circuitry 519 to close 3-way valves 508, 509, and/or 515 and/or shut off pump 510 based on data collected by the tracker or optical sensor 514 during monitoring.

In some implementations, a pressure sensor 511 and/or flow sensor 512 can be included in the fluidic system to monitor the fluidic connections between compartments and provide feedback to the processor 520 regarding adjusting the speed of pump 510 and/or pump 516.

It is to be understood that the components used in the compartments of the manifold implementation, including the purification components, the target biomolecular product, the measurable molecular reporter, the binding buffer, the washing buffer, and the elution buffer, can be any known reagents used in the production and/or purification of a target biomolecular product, including the reaction components used within the cartridge implementation.

1. A portable device for automated production of a purified target biomolecular product, the portable device comprising:
a purification compartment configured to contain purification components and to receive a raw biomolecular products mixture comprising a target biomolecular product, the purification components comprising an affinity matrix for binding the target biomolecular product;
a wash buffer compartment configured to contain a washing buffer comprising wash components;
an elution buffer compartment configured to contain an elution buffer comprising elution components;
fluidic channels configured to successively introduce into the purification compartment:
the raw biomolecular products mixture to allow binding of the target biomolecular product to the affinity matrix and produce a biomolecular product mixture comprising a bound target biomolecular product;
the washing buffer to wash the biomolecular product mixture and produce a waste mixture and a washed biomolecular product mixture comprising the bound target biomolecular product; and
the elution buffer to subject the washed biomolecular product mixture to elution and produce the purified target biomolecular product;
a fluidic system comprising a pump to supply the raw biomolecular products mixture, the washing buffer and the elution buffer to the purification compartment; and
a processor operatively connected to the fluidic system via electronic circuitry to control operation of the fluidic system.

2. The portable device of claim 1, wherein the fluidic system comprises valves that enable selective fluid communication of the wash buffer compartment and the elution buffer compartment with the purification compartment via the processor.

3. The portable device of claim 2, wherein the selective fluid communication is in accordance with pre-determined time points of a pre-determined production protocol.

4. The portable device of any one of claims 1 to 3, wherein the fluidic channels are etched, carved, embossed or molded in a manifold plate.

5. The portable device of claim 4, wherein the wash buffer compartment and the elution buffer compartment are each in fluid communication with a corresponding one of the fluidic channels via a corresponding fluidic port provided in the manifold plate.

6. The portable device of claim 4 or 5, wherein the manifold plate comprises a compartments-receiving section, and at least one of the wash buffer compartment and the elution buffer compartment is coupled to the manifold plate via the compartments-receiving section.

7. The portable device of any one of claims 4 to 6, wherein the manifold plate comprises two etched sheets superposed to one another to obtain the fluidic channels.

8. The portable device of claim 7, wherein the two etched sheets are etched acrylic sheets bonded together with a pressure adhesive, pressure controlled lamination, or temperature controlled lamination.

9. The portable device of any one of claims 4 to 8, wherein at least one of the purification compartment, the wash buffer compartment and the elution buffer compartment is removably coupled to the manifold plate via a corresponding fluidic port.

10. The portable device of any one of claims 1 to 3, wherein the fluidic channels are made of tubing.

11. The portable device of any one of claims 1 to 10, further comprising a reaction compartment comprising reaction components configured to produce the raw biomolecular products mixture comprising the target biomolecular product.

12. The portable device of claim 11, further comprising a heating system operatively connected to the reaction compartment and the processor to provide heat to the reaction compartment during the production of the raw biomolecular products mixture.

13. The portable device of claim 11 or 12, further comprising a cooling system operatively connected to the processor and to at least one of the purification compartment and the reaction compartment.

14. The portable device of any one of claims 11 to 13, wherein the reaction compartment further contains a measurable molecular reporter produced concomitantly with the target biomolecular product.

15. The portable device of claim 14, wherein the reaction components are cell-free reaction components.

16. The portable device of claim 15, wherein the cell-free reaction components are freeze-dried cell-free (FDCF) reaction components activable upon rehydration to produce the raw biomolecular products mixture.

17. The portable device of claim 15 or 16, wherein the cell-free reaction components enable production of RNA, purification of RNA, amplification of DNA, and/or purification of DNA.

18. The portable device of claim 14, wherein the reaction components comprise DNA and/or RNA coding for the target biomolecular product that is provided as dried pellets.

19. The portable device of any one of claims 14 to 18, further comprising an optical tracker in optical communication with the reaction compartment for monitoring the production of the raw biomolecular product via the production of the measurable molecular reporter.

20. The portable device of claim 19, wherein the optical tracker is at least one of a colorimetric tracker, a fluorescent tracker, and a UV tracker.

21. The portable device of claim 19 or 20, wherein the optical tracker is configured to signal the processor when the production of the measurable molecular reporter has reached a given threshold to indicate when to transfer the raw biomolecular products mixture to the purification compartment.

22. The portable device of any one of claims 19 to 21, wherein the measurable molecular reporter comprises a LacZ reporter gene, and the optical tracker is configured to monitor the production of the raw biomolecular product via the production of the LacZ reporter gene by measuring absorbance at a wavelength of approximately 570 nm.

23. The portable device of any one of claims 19 to 21, wherein the molecular reporter is a fluorescent reporter, and the fluorescent reporter is at least one of green fluorescent protein (GFP), enhanced green fluorescent protein (EGFP), mCherry and red fluorescent protein (RFP).

24. The portable device of any one of claims 14 to 18, further comprising at least one of an enzymatic tracker, an affinity-based tracker, and an electrochemical tracker.

25. The portable device of any one of claims 1 to 24, further comprising a binding buffer compartment in fluid communication with the purification compartment, the binding buffer compartment being configured to contain a binding buffer comprising buffer components.

26. The portable device of any one of claims 1 to 25, further comprising a waste compartment in fluid communication with the purification compartment, the waste compartment being configured to receive waste components from the purification compartment.

27. The portable device of any one of claims 1 to 26, further comprising a product dispensing compartment in fluid communication with the purification compartment, the product dispensing compartment being configured to receive the purified target biomolecule product from the purification compartment.

28. The portable device of any one of claims 1 to 27, wherein at least one of the wash components and the elution components are freeze-dried and activable upon rehydration.

29. The portable device of any one of claims 1 to 28, wherein the purification components are freeze-dried and activable upon rehydration with the raw biomolecular products mixture.

30. The portable device of any one of claims 1 to 29, wherein the affinity matrix comprises at least one of chitin-binding beads, nickel-nitrilotriacetic acid (Ni-NTA) beads, protein-A beads, amylose resin, agarose, and cellulose matrices, or combination thereof.

31. The portable device of any one of claims 1 to 30, wherein the elution components comprise an enzymatic cleavage component configured to release the target biomolecular product from the affinity matrix.

32. The portable device of claim 31, wherein the enzymatic cleavage component is a TEV protease or a chitin-binding/GST-tag/TEV Protease (S219V) chimera.

33. The portable device of any one of claims 1 to 30, wherein the elution components comprise a self-cleaving initiator component configured to release the target biomolecular product from the affinity matrix.

34. The portable device of claim 33, wherein the self-cleaving initiator component comprises at least one of dithiothreitol (DTT), 2-mercaptoethanol, cysteine, and hydroxylamine.

35. The portable device of any one of claims 1 to 30, wherein the elution components comprise a chemical agent configured to release the target biomolecular product from the affinity matrix.

36. The portable device of claim 35, wherein the chemical agent comprises imidazole or Histidine.

37. The portable device of any one of claims 1 to 36, further comprising a tracing system operatively connected to the processor, the tracing system configured to assign a unique identifier to a dose of the purified target biomolecular product.

38. The portable device of any one of claims 1 to 37, further comprising an actuator operatively connected to the processor via the electronic circuitry to initiate fluid communication between the washing buffer compartment and the purification compartment, or between the elution buffer compartment and the purification compartment.

39. The portable device of any one of claims 1 to 38, wherein the purified target biomolecular product comprises a therapeutic drug, a vaccine, an antibody or antibody fragment, a protein-based reagent, or an antivenom.

40. The portable device of any one of claims 1 to 39, wherein the portable device is configured to collect a series of manufacturing metrics.

41. The portable device of claim 40, wherein the series of manufacturing metrics is storable in an immutable block chain ledger.

42. The portable device of claim 40 or 41, wherein the series of manufacturing metrics comprises at least one of a date, a reaction duration, a reaction temperature, an origin of the cartridge, a DNA sequence, a yield of the purified target biomolecular product, location of manufacturing, identity of user, and a quality metric.

43. The portable device of any one of claims 1 to 42, further comprising an on-site DNA synthesizer.

44. The portable device of any one of claims 1 to 43, wherein at least one of the washing buffer compartment, the elution buffer compartment, and the purification compartment is an individual tube or vial.

45. The portable device of claim 1, wherein the washing buffer compartment, the elution buffer compartment, and the purification compartment are sealed and segmented compartments and are provided as part of a cartridge.

46. The portable device of claim 45, wherein the sealed and segmented compartments are configured to be punctured.

47. The portable device of claim 46, further comprising a reaction compartment comprising reaction components configured to produce the raw biomolecular products mixture comprising the target biomolecular product.

48. The portable device of claim 47, wherein the reaction compartment is configured to be punctured upon closing a lid of the portable device, loading the cartridge or activation of an actuator.

49. The portable device of any one of claims 46 to 48, wherein the sealed and segmented compartments are punctured simultaneously or according to a predetermined order.

50. The portable device of any one of claims 1 to 49, further comprising a pressure sensor operatively connected to the processor to monitor a pressure within at least one of the washing buffer compartment, the washing buffer compartment, and the purification compartment, and/or within the fluidic channels extending therebetween.

51. The portable device of any one of claims 1 to 50, further comprising a flow sensor operatively connected to the processor to monitor a flow rate between the washing buffer compartment and the purification compartment, or between the elution buffer compartment and the purification compartment.

52. The portable device of any one of claims 1 to 51, wherein the processor is separated from wet components of the portable device to prevent passage of liquid to the processor, the wet components comprising at least one of the compartments.

53. The portable device of any one of claims 1 to 52, further comprising a second optical tracker in optical communication with the fluidic channels for monitoring fluid circulation within the fluidic channels.

54. A method for automated production of a purified target biomolecular product from a raw biomolecular products mixture comprising a target biomolecular product, the method comprising:
activating a portable device to initiate the automated production of the purified target biomolecular product, the portable device comprising:
a purification compartment comprising purification components configured to bind the target biomolecular product;
a fluidic system configured to supply the raw biomolecular products mixture to the purification compartment; and
a processor to control the automated production of the purified target biomolecular product; and
subjecting the raw biomolecular products mixture to purification to produce the purified biomolecular product.

55. The method of claim 54, wherein subjecting the raw biomolecular products mixture to purification comprises:
supplying the raw biomolecular products mixture to the purification compartment to produce a biomolecular product mixture comprising a bound target biomolecular product;
transferring a wash buffer to the purification compartment to wash the biomolecular product mixture and produce a waste mixture and a washed biomolecular product mixture comprising the bound target biomolecular product;
removing the waste product from the purification compartment; and
transferring an elution buffer to the purification compartment to elute the purified target biomolecular product.

56. The method of a claim 55, wherein the portable device further comprises a washing buffer compartment configured to contain the wash buffer.

57. The method of any one of claims 55 and 56, wherein the portable device further comprises an elution buffer compartment configured to contain the elution buffer.

58. The method of any one of claims 54 to 57, further comprising synthesizing the raw biomolecular products mixture comprising a target biomolecular product and a measurable molecular reporter produced concomitantly with the target biomolecular product in a reaction compartment prior to supplying the raw biomolecular products mixture to the purification compartment.

59. The method of claim 58, further comprising activating the fluidic system to pump air into the reaction compartment during synthesis of the raw biomolecular products mixture.

60. The method of claim 58 or 59, further comprising monitoring a temperature of the reaction components in the reaction compartment via a temperature sensor.

61. The method of claim 60, further comprising adjusting the temperature in the reaction compartment with a heating system and/or a cooling system.

62. The method of claim 61, wherein adjusting the temperature in the reaction compartment is performed to maintain reaction conditions according to a pre-determined protocol.

63. The method of any one of claims 58 to 62, further comprising monitoring the production of the raw biomolecular product via the production of the measurable molecular reporter.

64. The method of claim 63, wherein monitoring of the production of the raw biomolecular product via the production of the measurable molecular reporter is performed to produce a single dose of the purified target biomolecular product or a plurality of doses thereof.

65. The method of claim 63 or 64, wherein monitoring the production of the raw biomolecular product via the production of the measurable molecular reporter allows for batch number traceability.

66. The method of any one of claims 54 to 59, wherein the portable device further comprises a product dispenser compartment.

67. The method of claim 66, further comprising monitoring a temperature of the purified target biomolecular product in the product dispenser compartment via a temperature sensor.

68. The method of claim 67, further comprising adjusting the temperature in the product dispenser compartment with a heating system and/or a cooling system.

69. The method of claim 68, wherein adjusting the temperature in the product dispenser compartment is performed to maintain stabilizing conditions according to a pre-determined validation protocol.

70. The method of any one of claims 54 to 69, further comprising transferring a binding buffer from a binding buffer compartment to the purification compartment to equilibrate the purification components prior to supplying the raw biomolecular products mixture to the purification compartment.

71. The method of any one of claims 55 to 70, further comprising monitoring a pressure within at least of the washing buffer compartment, the elution buffer compartment, the purification compartment, and/or within the fluidic channels extending therebetween.

72. The method of any one of claims 55 to 71, further comprising monitoring a flow rate of the components between the washing buffer compartment and the purification compartment, or between the elution buffer compartment and the purification compartment.

73. A cartridge for use in a device for automated production of a purified target biomolecular product, the cartridge comprising:
a reaction compartment comprising a raw biomolecular products mixture comprising a target biomolecular product and a measurable molecular reporter; and
a purification compartment comprising purification components, the purification compartment being configured to successively receive therein:
the raw biomolecular products mixture to allow binding of the target biomolecular product to produce a bound target biomolecular product;
a washing buffer to wash the bound target biomolecular product and produce a waste mixture and a washed biomolecular product mixture; and
an elution buffer to subject the washed biomolecular product mixture to elution and release the target biomolecular product from the purification components to produce the purified target biomolecular product.

74. The cartridge of claim 73, wherein the purification components comprise an affinity matrix for binding the target biomolecular product.

75. The cartridge of claim 73 or 74, further comprising a washing buffer compartment configured to contain the washing buffer.

76. The cartridge of any one of claims 73 to 75, further comprising an elution buffer compartment configured to contain the elution buffer.

77. The cartridge of any one of claims 73 to 76, wherein the reaction compartment comprises reaction components to produce the raw biomolecular products mixture comprising the target biomolecular product and the measurable molecular reporter produced concomitantly with the target biomolecular product.

78. A portable device for automated production of a purified target biomolecular product, the portable device comprising:
compartments coupled to a compartments-receiving section, the compartments comprising:
a wash buffer compartment configured to contain wash components;
an elution buffer compartment configured to contain elution components; and
a purification compartment configured to contain purification components;
a fluidic system for successively providing a raw biomolecular products mixture, the washing buffer, and the elution buffer to the purification compartment; and
a processor operatively connected to the fluidic system via electronic circuitry to control operation of the fluidic system.

79. A portable device for automated production of a purified target biomolecular product, the portable device comprising:
a cartridge comprising:
a purification compartment configured to contain purification components and to receive a raw biomolecular products mixture comprising a target biomolecular product for binding the target biomolecular product;
a wash buffer compartment configured to contain a washing buffer comprising wash components; and
an elution buffer compartment configured to contain an elution buffer comprising elution components;
a fluidic system for successively providing a raw biomolecular products mixture, the washing buffer, and the elution buffer to the purification compartment; and
a processor powerable by a power source and operatively connected to the fluidic system for controlling the automated production of the purified target biomolecular product.
